(19) United States
(12) Patent Application Publication
(10) Pub. No.: US 2009/0240108 A1
(43) Pub. Date: Sep. 24, 2009

(54) CAPSULE ENDOSCOPY SYSTEM AND METHOD OF CONTROLLING OPERATION OF CAPSULE ENDOSCOPE

(75) Inventors: Kunimasa SHIMIZU, Minato-ku (JP); Kenichi OTANI, Ashigarakami-gun (JP); Naoto KINJO, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/406,383

(22) Filed: Mar. 18, 2009

(30) Foreign Application Priority Data
Mar. 21, 2008 (JP) 2008-073534

(51) Int. Cl. A61B 1/045

(52) U.S. Cl. 600/109

(57) ABSTRACT

A capsule endoscope captures an image from an imaging field as it travels through a tract of a patient and, at the same time, measures subject distances to multiple points in the imaging field. The capsule endoscope sends the captured image and information on the multi-point distances wirelessly to a data transceiver that the patient carries about. With reference to the multi-point distance information, a check zone of a limited distance range is determined in the imaging field, and the check zone is divided into small blocks. Image characteristic values are extracted from image data of each individual small block, and compared with the image characteristic values of other small blocks, to examine similarity between the small blocks. Those small blocks which are less similar to other small blocks are considered to constitute an area of concern, such as a lesion.

### FIELD OF THE INVENTION

The present invention relates to a capsule endoscopy system for making medical diagnoses by means of endoscopic images captured by a capsule endoscope. The present invention relates also to a method of controlling operation of the capsule endoscope.

### BACKGROUND OF THE INVENTION

Endoscopy with a capsule endoscope has recently been put into practical use. The capsule endoscope has its components, including an imaging device and an illumination light source, integrated into a micro capsule. A patient first swallows the capsule endoscope so that the imaging device captures images from interior of the patient, i.e. internal surfaces of patient's tracts, while the light source is illuminating those surfaces. Image data captured by the imaging device is transmitted as a radio signal to a receiver that the patient carries about. The image data is sequentially recorded on a storage medium like a flash memory, which is provided in the receiver. During or after the endoscopy, the image data is transmitted to an information managing apparatus like a workstation, where endoscopic images are displayed on a monitor for the sake of image interpretation and diagnosis.

The capsule endoscope captures images a given number of times per unit time, e.g. at a frame rate of 2 fps (frame per second). Since the capsule endoscope takes more than eight hours or so to complete capturing the images from each patient, the volume of the image data that have been taken and stored in the receiver gets huge at the end of each session of the endoscopy. So it takes a very long time and consumes much labor for the doctor to interpret all of the captured endoscopic images for the sake of diagnosis. For this reason, there has been a demand for reducing such images that are unnecessary for the diagnosis to the minimum, while capturing as many images from an important site for the diagnosis as possible. To meet the demand, such a capsule endoscope has been suggested that captures images according to a predetermined time schedule, for example, in JPA 2005-193066.

The above-mentioned prior art discloses an example, wherein the capsule endoscope raises the frame rate as it goes through an area of concern, like where there is a lesion, and lowers the frame rate after it goes past the area of concern. However, this prior art does not specify any concrete device for determining the area of concern, so it is still difficult to interpret the captured images in detail with respect to the area of concern.

In order to determine the area of concern, it may for example be possible to compare present information that the capsule endoscope obtains at present from the patient with past information on the patient. The present information may include endoscopic images and positional information on the positions where these images were taken, whereas the past information may be information on a past diagnosis for the patient, including an image of an area of concern and information on the position of the area of concern. Instead of the past information on the patient, it is possible to compare the present information with general information on medical cases, such as an image exemplar representative of a case of disease, to determine an area of concern. This method is applicable to a patient who gets the endoscopy for the first time.

Because the above-described methods of determining the area of concern need the information on the past diagnoses or on general cases, it is impossible to determine the area of concern without such information. Even if there is the diagnostic information or the general case information, if the information was obtained by a different kind of endoscope from the presently used endoscope, the difference between the endoscopes can induce such a problem that images taken at the same portion by the present endoscope and the other kind of endoscope have different features from each other. In that case, it is hard to determine the area of concern exactly.

Moreover, since the general case information or images are representative data sorted out from an enormous database built up through many diagnoses done in the past, an individual endoscopic image taken from a lesion of a patient is not always similar to the case image representative of the corresponding case. If the endoscopic images taken from the lesion of the patient are not similar to the corresponding case image, it is impossible to identify the lesion as an area of concern.

### SUMMARY OF THE INVENTION

In view of the foregoing, a primary object of the present invention is to provide a capsule endoscopy system using a capsule endoscope and a method of controlling operation of the capsule endoscope, whereby an area of concern that may contain a lesion or the like is determined exactly without the need for the diagnostic information or the general case information.

A capsule endoscopy system of the present invention comprises a judging device that analyzes each endoscopic image immediately after it is obtained by an imaging device of a capsule endoscope, to judge by the result of analysis whether the endoscopic image contains any area of concern that has different image characteristics from surrounding areas. The judging device is mounted at least in one of the capsule endoscope, a portable apparatus and an information managing apparatus, wherein the capsule endoscope is swallowed by a test body, captures endoscopic images of internal portions of the test body through the imaging device, and sends the endoscopic images wirelessly. The portable apparatus is carried about by the test body, and receives the endoscopic images from the capsule endoscope and stores the received endoscopic images. The information managing apparatus stores and manages the endoscopic images that are transferred from the portable apparatus.

Preferably, the capsule endoscopy system of the present invention further comprises a control command generator for generating control commands for controlling operations of respective members of the capsule endoscope on the basis of a result of the judgment by the judging device, and an operation controller mounted in the capsule endoscope, for controlling operations of the respective members of the capsule endoscope in accordance with the control commands. The control command generator is mounted at least in one of the capsule endoscope, the portable apparatus and the information managing apparatus.

According to a preferred embodiment, the judging device divides each endoscopic image into a plurality of segments, examines similarity among these segments, and judges that an area of concern exists in the endoscopic image when there are some segments that bear relatively low similarities to other segments of the endoscopic image. The judging device detects image characteristic values from the respective segments, calculates differences in the image characteristic values between the respective segments, and estimates the similarity between the segments by comparing the calculated differences with predetermined threshold values.

According to another preferred embodiment, the judging device examines similarity between the latest endoscopic image obtained from the capsule endoscope and the preceding image obtained immediately before from the capsule endoscope. The judging device judges that an area of concern exists in the latest endoscopic image if the latest endoscopic image is not similar to the preceding image and the judging device has judged that no area of concern exits in the preceding image, or if the latest endoscopic image is similar to the preceding image and the judging device has judged an area of concern exits in the preceding image.

To estimate similarity between the segments of the latest endoscopic image and corresponding segments of the preceding image Preferably, the judging device preferably divides each endoscopic image into a plurality of segments, and judges that the latest endoscopic image is not similar to the preceding image when there are some segments that bear relatively low similarities to the corresponding segments of the preceding image. More preferably, the judging device detects image characteristic values from the respective segments of the latest and preceding endoscopic images, calculates differences in the image characteristic values between each individual segment of the latest endoscopic image and the corresponding segment of the preceding image, and estimates the similarity between each couple of the corresponding segments of the latest and preceding images by comparing the calculated differences with predetermined threshold values.

According to another preferred embodiment, the capsule endoscope comprises a multi-point ranging device for measuring distances from the capsule endoscope to a plurality of points of a subject in a present imaging field of the imaging device, and the judging device executes a cropping process for cutting a zone of a limited subject distance range out of each endoscopic image on the basis of the distances measured by the multi-point ranging device, and analyzes image data of the zone of the endoscopic image to judge whether any area of concern exits in the zone.

According to a further preferred embodiment, the control command generating device generates a first control command for driving the capsule endoscope in a regular imaging mode when the judging device judges that no area of concern exits, whereas the control command generating device generates a second control command for driving the capsule endoscope in a special imaging mode when the judging device judges that an area of concern exits, so the capsule endoscope may capture detailed images of the area of concern in the special mode.

The capsule endoscope of the capsule endoscopy system of the present invention may comprise at least two imaging devices facing different directions from each other and a direction sensor for detecting attitude and traveling direction of the capsule endoscope. In this embodiment, the control command generator determines respective facing directions of the imaging devices on the basis of the detected attitude and traveling direction of the capsule endoscope, and generates a control command for driving a forward one of the imaging devices, which presently faces forward in the traveling direction, in a regular imaging mode. When the judging device judges that an area of concern exits in an endoscopic image as captured by the forward imaging device, the control command generator generates a second control command for driving at least one of other imaging devices than the forward imaging device in a special imaging mode for capturing detailed images of the area of concern.

A method of controlling operations of a capsule endoscope that is swallowed by a test body, to capture endoscopic images of internal portions of the test body and output the endoscopic images wirelessly, wherein the method comprising steps of:

analyzing each endoscopic image immediately after it is obtained by the capsule endoscope;

judging by a result obtained by the analyzing step whether the endoscopic image contains any area of concern that has different image characteristics from surrounding areas;

generating control commands for controlling operations of respective members of the capsule endoscope on the basis of a result of the judging step; and

controlling operations of the respective members of the capsule endoscope in accordance with the control commands.

According to the present invention, since an area of concern, such as a lesion, generally has different features from its surrounding area, the endoscopic image itself is analyzed each time it is captured by the capsule endoscope, and the judgment about the presence of any area of concern is made merely by the result of analysis of the endoscopic image itself, without the need for the past diagnostic information on the patient or the case information on the general cases. Since the area of concern is determined in a real time fashion during the capsule endoscopy, it is possible to capture detailed images of the area of concern by switching the capsule endoscope to the special imaging mode as soon as the area of concern is discovered. Moreover, it comes to be possible to identify such a lesion that is not similar to the case information. Because the present invention does not need the diagnostic information or the case information, it is also unnecessary to consider differences between the capsule endoscopes used for obtaining the diagnostic information or the case information, on one hand, and the capsule endoscope used for the present endoscopy.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several views, and wherein:

FIG. 1 is a schematic diagram illustrating a capsule endoscopy system according to an embodiment of the present invention;

FIG. 2 is a sectional view illustrating an interior of a capsule endoscope of the capsule endoscopy system;

FIG. 3 is an explanatory diagram illustrating a multi-point ranging process for ranging an imaging field;

FIG. 4 is a block diagram illustrating an electric structure of the capsule endoscope;

FIG. 5 is a block diagram illustrating an electric structure of a data transceiver;

FIG. 6 is an explanatory diagram illustrating a cropping process for cutting image data of a check zone out of an image frame;

FIGS. 7A and 7B are explanatory diagrams illustrating a distance range of the check zone relative to the capsule endoscope;

FIG. 8 is an explanatory diagram illustrating a judging process for judging whether any area of concern exists in the check zone;

FIG. 9 is a flowchart illustrating a sequence of an imaging mode selection process;

FIG. 10 is an explanatory diagram illustrating an example of an imaging condition table;

FIG. 11 is a block diagram illustrating an electric structure of a workstation;

FIG. 12 is a flowchart illustrating an overall operation of the capsule endoscopy system;

FIG. 13 is a flowchart illustrating an imaging mode selection process according to a second embodiment of the present invention;

FIG. 14 is a sectional view illustrating an interior of a capsule endoscope according to a third embodiment;

FIG. 15 is an explanatory diagram illustrating the third embodiment, wherein an optical axis of an objective lens system is veered toward an area of concern in a special imaging mode;

FIG. 16 is a sectional view illustrating an interior of a capsule endoscope according to a fourth embodiment;

FIGS. 17A and 17B are explanatory diagrams illustrating the fourth embodiment, wherein an imaging device that faces forward in the traveling capsule endoscope captures images in a regular imaging mode, whereas another imaging device that faces rearward is driven in a special imaging mode;

FIG. 18 is an explanatory diagram illustrating a fifth embodiment;

FIG. 19 is a block diagram illustrating an electric structure of a capsule endoscope that analyzes image data and generates control commands by itself; and

FIG. 20 is a block diagram illustrating an electric structure of an endoscopy system, wherein a workstation analyzes image data from a capsule endoscope and generates control commands for the capsule endoscope.

### DESCRIPTION OF THE PREFERRED EMBODIMENTS

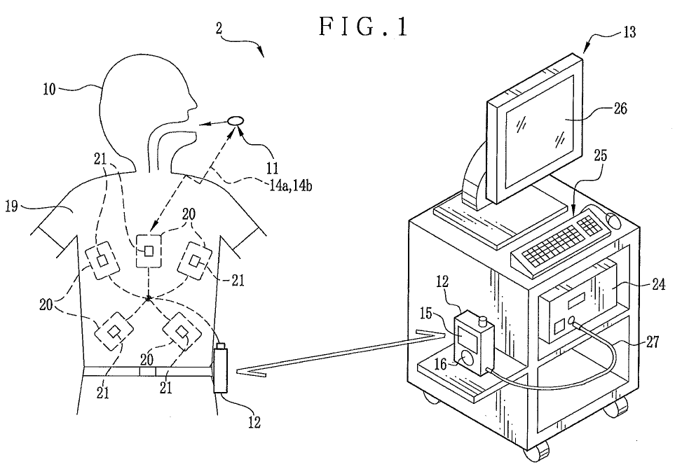

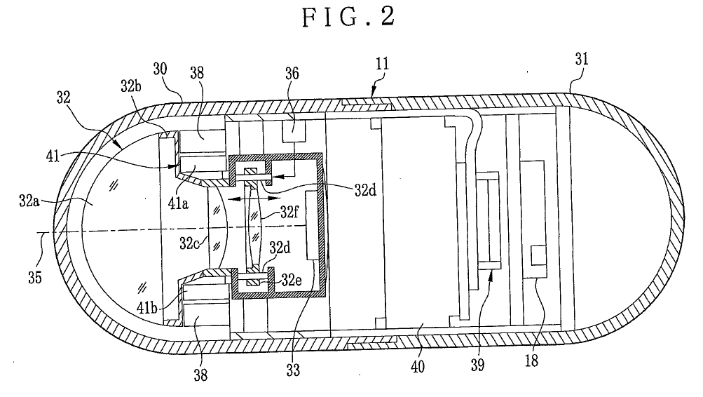

As shown in FIGS. 1 and 2, an endoscopy system 2 consists of a capsule endoscope 11 that is swallowed by a patient or test body 10, a portable data transceiver 12 carried about by the patient 10, and a workstation 13 that takes up endoscopic images as captured by the capsule endoscope 11 and displays the endoscopic images for a doctor to interpret them. In the capsule endoscopy system 2, image data of the latest endoscopic image as captured by the capsule endoscope 11 is wirelessly transmitted to the data transceiver 12, so the data transceiver 12 analyzes the endoscopic image to check if any concerned part like a lesion exits in the image. If there is, more detailed endoscopic images of the concerned part is captured by the capsule endoscope 11 after setting it to a special imaging mode.

The capsule endoscope 11 captures images from internal walls of tracts, e.g. bowels, of the patient 10, to send data of the captured images to the data transceiver 12 sequentially as a radio wave 14a. The capsule endoscope 11 also receives control command as a radio wave 14b from the data transceiver 12, and operates according to the control command.

The data transceiver 12 is provided with a liquid crystal display (LCD) 15 for displaying various setup screens and an operating section 16 for setting up the data transceiver 12 on the setup screens. The data transceiver 12 receives and stores the image data as transmitted from the capsule endoscope 11 on the radio wave 14a. The data transceiver 12 also analyzes the latest image data as obtained from the capsule endoscope 11, to decide the imaging conditions of the capsule endoscope 11 by the result of the analysis. That is, the data transceiver 12 decides which imaging mode the capsule endoscope 11 is to be set to, and produces a control command for setting the capsule endoscope 11 to the decided imaging mode. The control command is sent from the data transceiver 12 to the capsule endoscope 11 on the radio wave 14b.

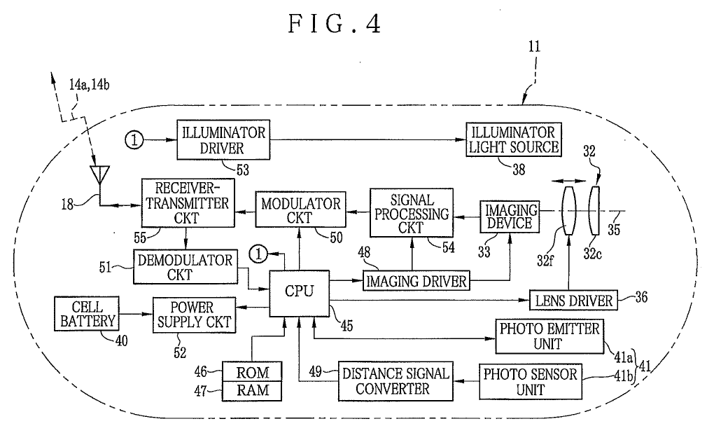

The transmission of the radio waves 14a and 14b between the capsule endoscope 11 and the data transceiver 12 is carried out by way of antennas 18 and 20, wherein the antenna 18 is mounted in the capsule endoscope 11, as shown in FIGS. 2 and 4, whereas the antennas 20 are mounted on a shield shirt 19 that the patient 10 wears. Each of the antennas 20 has an electric field strength sensor 21 built therein for measuring the field strength of the radio wave 14a from the capsule endoscope 11.

The capsule endoscope 11 has a regular imaging mode for obtaining image data of ordinary endoscopic images, and the special imaging mode for obtaining image data of high-definition endoscopic images. The capsule endoscope 11 is set to different imaging conditions in the special imaging mode from the regular imaging mode. Concretely, in the special imaging mode, the frame rate is raised, and the zooming magnification (field of view) and the exposure value (the shutter speed and the illumination light volume) are changed step by step at each exposure.

The workstation 13 is provided with a processor 24, operating members 25, including a keyboard and a mouse, and an LCD monitor 26. The first processor 24 is connected to the data transceiver 12, for example, through a USB cable 27, to exchange data. The first processor 24 may be connected to the data transceiver 12 through wireless communication like infrared communication. During or after the endoscopy with the capsule endoscope 11, the processor 24 takes up the image data from the data transceiver 12, accumulates and manages the image data for individual patients, and produces TV images from the image data to display the TV images on the LCD 26.

As shown in FIG. 2, the capsule endoscope 11 has a transparent front casing 30 and a rear casing 31 that is mated to the front casing 30 to form a water-tight room inside these casings 30 and 31. The casings 30 and 31 have a cylindrical shape with one end open and the other end closed. The closed ends of the casings 30 and 31 are substantially semispherical. In the room inside the casings 30 and 31, an objective lens system 32 and an imaging device 33, such as a CCD image sensor or a CMOS image sensor, are mounted. While the capsule endoscope 11 is inside the patient 10, the objective lens system 32 forms an optical image of an internal body part or site of the patient 10 on an image pickup surface of the imaging device 33, so the imaging device 33 outputs an analog images signal corresponding to the optical image. Designated by 35 is an optical axis of the objective lens system 32.

The objective lens system 32 is composed of a transparent convex optical dome 32a, a first lens holder 32b, the first lens system 32c, guide rods 32d, a second lens holder 32e, and a second lens 32f. The optical dome 32a is placed in the semispherical end of the front casing 30. The first lens holder 32b is mounted to a rear end of the optical dome 32a, and is tapered off rearwards. The first lens system 32c is secured to the first lens holder 32b.

The guide rods 32d are screw rods, which are mounted to the rear end of the first lens holder 32b in parallel to the optical axis 35. The second lens holder 32e has female screw holes, through which the guide rods 32d are threaded, so that the second lens 32f moves in parallel to the optical axis 35 as the guide rods 32d is turned by a lens driver 36 that is constituted of a stepping motor and other minor elements. With the parallel movement of the second lens 32f to the optical axis 35, the zooming magnification (focal length) of the objective lens system 32 varies, and thus the field of view (imaging field) of the objective lens system 32 varies correspondingly. The lens driver 36 varies zooming magnification of the objective lens system 32 so that each image is captured at a given zooming magnification and in a given field of view, which are designated by the control command.

Inside the casings 30 and 31, an antenna 18 for sending and receiving the radio waves 14a and 14b, an illumination light source 38 for illuminating the body parts, an electric circuit board 39 having various electronic circuits mounted thereon, a button cell 40 and a multi-point ranging sensor 41 are mounted.

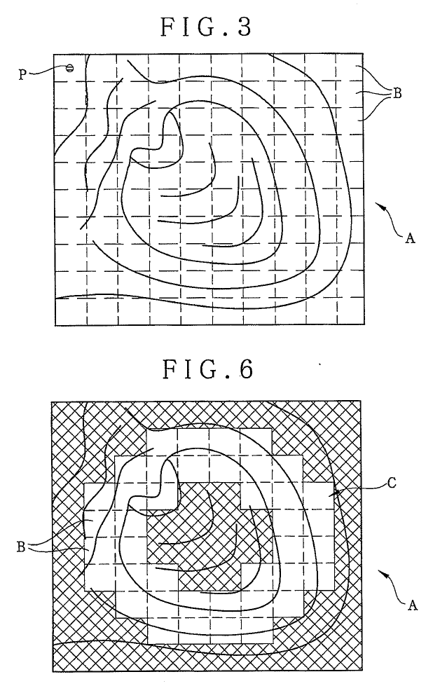

The multi-point ranging sensor 41 is an active sensor that consists of a photo emitter unit 41a and a photo sensor unit 41b. Each time the capsule endoscope 11 captures an endoscopic image, the multi-point ranging sensor 41 measures respective distances from the capsule endoscope 11 to a plurality of points of a subject, i.e. an internal body portion, which corresponds to the captured endoscopic image. As shown in FIG. 3, the multi-point ranging sensor 41 divides the imaging field A of the capsule endoscope 11 into a plurality of ranging blocks B, which are arranged in a matrix, and measures a distance to a representative point P of every block B. For example, the representative points P are located at respective centers of the ranging blocks B, although the point P is shown only in one of the ranging blocks B in FIG. 3, in order to avoid complicating the drawing.

The photo emitter unit 41a projects a near infrared ray toward the representative point P of one ranging block B to another in a predetermined sequence. A conventional method is usable for projecting the near infrared ray toward the respective representative points P. For example, the photo emitter unit 41a is turned around at least in a direction: a yaw direction that is around a vertical axis of the capsule endoscope 11, or a pitch direction that is around a horizontal axis of the capsule endoscope 11, thereby to scan the near infrared ray two-dimensionally across the imaging field A. Note that the ray projected from the photo emitter unit 41a is not limited to the near infrared ray, but may be a ray of another wavelength range insofar as it does not affect the imaging.

The near infrared ray is projected from the photo emitter unit 41a toward the representative point P, and is reflected from the representative point P and is received on the photo sensor unit 41b. The photo sensor unit 41b is for example a position sensitive detector (PSD). As known in the art, see for example JPA 2007-264068, the PSD outputs an electric signal as it receives the ray reflected from the representative point P, and the magnitude of the electric signal corresponds to the distance from the capsule endoscope 11 to the representative point P. So the electric signal output from the photo sensor unit 41b will be referred to as a distance measuring signal. Based on the distance measuring signal, the distance from the capsule endoscope 11 to the representative point P is calculated. Note that a distance signal conversion circuit 49 (see FIG. 4) converts the distance measuring signal to a distance signal that represents the distance from the capsule endoscope 11 to the representative point P.

The photo emitter unit 41a projects the near infrared ray sequentially toward the respective representative points P of the ranging blocks B, so the photo sensor unit 41b sequentially receives the ray reflected from each of the representative points P and outputs the distance measuring signals that represent respective distances from the capsule endoscope 11 to the representative points P. This way, the multi-point ranging is done to measure the distances to the representative points P of the respective ranging blocks B of the imaging field A.

In FIG. 4, a CPU (operation control device) 45 controls the overall operation of the capsule endoscope 11. The CPU 45 is connected to the lens driver 36, the multi-point ranging sensor 41, a ROM 46, a RAM 47, an imaging driver 48, the distance signal conversion circuit 49, a modulator circuit 50, a demodulator circuit 51, a power supply circuit 52 and an illuminator driver 53.

The ROM 46 stores various programs and data for controlling the operation of the capsule endoscope 11. The CPU 45 reads out necessary programs and data from the ROM 46 and develops them on the RAM 47, to work out the read program sequentially. The RAM 47 temporarily memorizes data on the imaging conditions, including a frame rate, a zooming magnification (view field) and an exposure value (a shutter speed and a light volume), as designated by the control command from the data transceiver 12.

The imaging driver 48 is connected to the imaging device 33 and a signal processing circuit 54. The imaging driver controls the operation of the imaging device 33 and the signal processing circuit 54 so as to make an exposure at the frame rate and the shutter speed, which are designed by the control command. The signal processing circuit 54 processes the analog image signal output from the imaging device 33, to convert the image signal to digital image data by means of correlated double sampling, amplification and analog-to-digital conversion. The signal processing circuit 54 also subjects the image data to gamma correction and other image processing.

The distance signal conversion circuit 49 is connected to the photo sensor unit 41b, and is supplied with the distance measuring signals from the photo sensor unit 41b. Then the distance signal conversion circuit 49 converts the respective distance measuring signals to the distance signals. The distance signal conversion circuit 49 may converts the distance measuring signal to the distance signal by means of a predetermined calculation formula or a data table or any other conventional method, so the detail of the conversion method will be omitted. The distance signals are fed to the CPU 45. When the CPU 45 receives a set of the distance signals that represent the distances to the respective representative points P in the imaging field A, the CPU 45 outputs each set of the distance signals as multi-point distance information on the imaging field A to the modulator circuit 50.

The modulator circuit 50 and the demodulator circuit 51 are connected to a receiver-transmitter circuit 55, which is connected to the antenna 18. The modulator circuit 50 modulates the digital image data from the signal processing circuit 54 and the multi-point distance information output from the CPU 45 to the radio wave 14a. That is, the image data and the multi-point distance information of the imaging field A from which the image data was obtained are modulated together into the radio wave 14a. The radio wave 14a is sent from the modulator circuit 50 to the receiver-transmitter circuit 55. The receiver-transmitter circuit 55 amplifies and band-pass filters the radio wave 14a, and then outputs the radio wave 14a to the antenna 18. The receiver-transmitter circuit 55 also amplifies and band-pass filters the radio wave 14b that is received on the antenna 18 from the data transceiver 12, and then outputs the radio wave 14b to the demodulator circuit 51. The demodulator circuit 51 demodulates the radio wave 14b to the original control command, and outputs the control command to the CPU 45.

The power supply circuit 52 supplies power of the cell 40 to respective components of the capsule endoscope 11. The illuminator driver 53 drives the illuminator light source 38 under the control of the CPU 45, so that each image is captured under the illumination light volume that is designated by the control command.

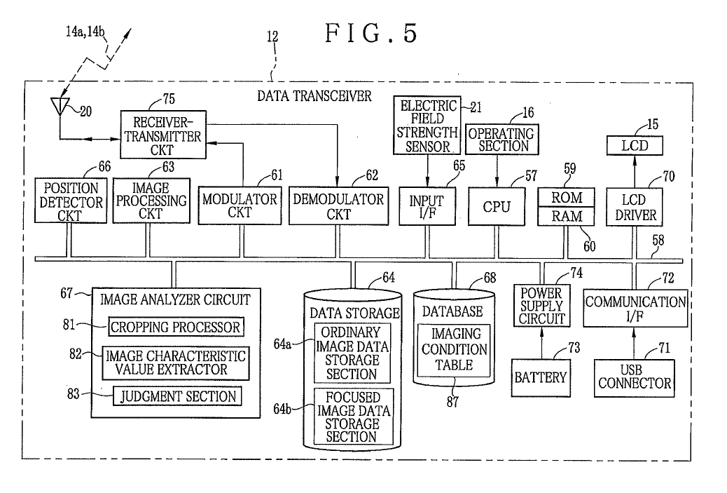

As shown in FIG. 5, a CPU 57 (control command production device) controls the overall operation of the data transceiver 12. A data bus 58 connects the CPU 57 to a ROM 59, a RAM 60, a modulator circuit 61, a demodulator circuit 62, an image processor circuit 63, a data storage 64, an input interface (I/F) 65, a position detector circuit 66, an image analyzer circuit (judging device) 67 and a database 68.

To the data bus 58 are also connected an LCD driver 70 for controlling the display on the LCD 15, a communication interface (I/F) 72 for a USB connector 71 to intermediate data exchange between the processor 24 and the data transceiver 12, and a power supply circuit 74 for supplying power of a battery 73 to respective components of the data transceiver 12.

The ROM 59 stores various programs and data for controlling the operation of the data transceiver 12. The CPU 57 reads out necessary programs and data from the ROM 59 and develops them on the RAM 60, to work out the read program sequentially. The CPU 57 also controls the respective components of the data transceiver 12 to operate in accordance with operational signals input through the operating section 16.

The modulator circuit 61 and the demodulator circuit 62 are connected to a receiver-transmitter circuit 75, which is connected to the antennas 20. The modulator circuit 61 modulates the control command to the radio wave 14b, and outputs the radio wave 14b to the receiver-transmitter circuit 75. The receiver-transmitter circuit 75 amplifies and band-pass filters the radio wave 14b from the demodulator circuit 61, and then outputs the radio wave 14b to the antennas 20. The receiver-transmitter circuit 75 also amplifies and band-pass filters the radio wave 14a that is received on the antennas 20 from the capsule endoscope 11, and then outputs the radio wave 14a to the demodulator circuit 62. The demodulator circuit 62 demodulates the radio wave 14a to the original image data and the multi-point distance information, and outputs the image data to the image processor circuit 63. The multi-point distance information is temporarily stored in the RAM 60 or the like.

The image processor circuit 63 processes the image data as demodulated by the demodulator circuit 62, and outputs the processed image data to the data storage 64 and the image analyzer circuit 67.

The data storage 64 is, for example, a flash memory having a memory capacity of 1 GB or so. The data storage 64 stores and accumulates the image data as being sequentially output from the image processor circuit 63. The data storage 64 has an ordinary image data storage section 64a and a focused image data storage section 64b. The ordinary image data storage section 64a stores image data obtained by the capsule endoscope 11 in the regular imaging mode, whereas the focused image data storage section 64b stores image data obtained by the capsule endoscope 11 in the special imaging mode.

The input interface 65 gets results of measurement from the electric field strength sensors 21, and outputs the results to the position detector circuit 66. The position detector circuit 66 detects a present position of the capsule endoscope 11 inside the patient 10 on the basis of the results of measurement of the electric field strength sensors 21, and outputs information on the detected position of the capsule endoscope 11, hereinafter referred to as imaging position data, to the data storage 64. The data storage 64 records the imaging position data in association with the image data from the image processor circuit 63. Since the method of detecting the position of the capsule endoscope 11 inside the test body on the basis of the field strength of the radio wave 14 from the capsule endoscope 11 is well known in the art, details of this method are omitted from the present description.

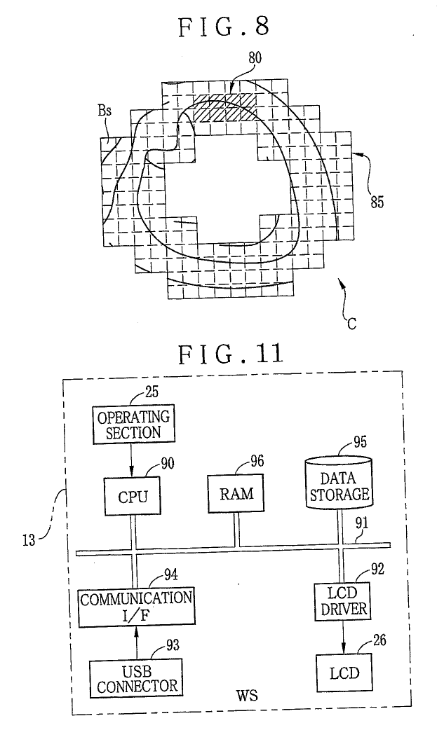

The image analyzer circuit 67 analyzes the image data of the latest image frame obtained by the capsule endoscope 11 as the image data of the latest image frame is fed from the image processor circuit 63, to judge whether the image frame contains any area of concern 80 (see FIG. 8) that has a different feature from its periphery and thus can be regarded a lesion or the like. The image analyzer circuit 67 is provided with a cropping processor 81, an image characteristic value extractor 82 and a judgment section 83.

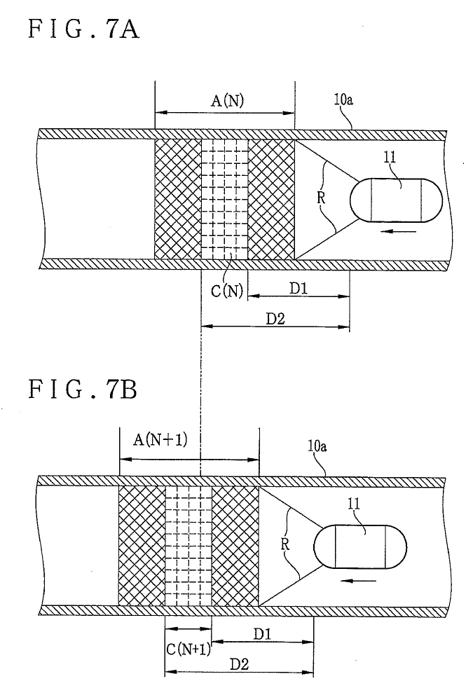

The cropping processor 81 reads out the multi-point distance information from the RAM 60 in correspondence to the image data from the image processor circuit 63, to process the image data for the cropping on the basis of the read multi-point distance information. Concretely, as shown in FIGS. 6, 7A and 7B, a zone of a limited distance range from the capsule endoscope 11: D1 to D2 (D1<D2), is defined to be a check zone C, and pixels in the check zone C are gained from the image frame. In other words, the judgment as to whether there is any area of concern 80 in the imaging field A is made only in the check zone C. Note that other areas than the check zone C in the imaging field A are hatched in FIGS. 6 and 7. A reference numeral 10a designates the body tract, and R designates the view field of the capsule endoscope 11 in FIG. 7.

The distance range D1 to D2 of the check zone C is so defined that all area in the body tract 10a will be checked over. Concretely, as shown in FIGS. 7A and 7B, the check zone C(N) in the imaging field A(N) of the N^{th} image frame adjoins or overlaps the next check zone C(N+1) in the imaging field A(N+1) of the (N+1)^{th} image frame, wherein N is a natural number.

The cropping processor 81 crops or cuts image data of the check zone C out of the image frame, and writes the cropped image data temporarily in the RAM 60 or the like. Limiting the zone of checking for an area of concern 80 allows checking the content in the limited image zone in detail, which helps finding an area of concern 80 like a lesion even if it is very small. Although it is possible to check the whole imaging field A in detail, it takes too much time. Besides, where the imaging field A(N) and the next imaging field A(N+1) overlap widely, wide area of the subject would be redundantly checked twice or more if the image analyzer circuit 67 checks the whole area of each image frame.

Moreover, limiting the zone of checking for an area of concern 80 to the predetermined distance range D1 to D2 of each imaging field A contributes to accurate judgment as to whether there is any lesion in the check zone. Because the surface condition and color of the internal surface of the test body, such as an inner wall of a tract, is normally similar or uniform in a limited area, it becomes easier to distinguish an abnormal portion like a lesion from the normal portion. In other words, the wider the target area of the subject, the wider variation appears even in its normal surface condition and color. So it becomes more difficult to distinguish the lesion from the normal portion.

The image characteristic value extractor 82 (see FIG. 5) reads out the cropped image data from the RAM 60, and extracts image characteristic values of the cropped image data. As shown in FIG. 8, the characteristic value extractor 82 divides the check zone C into a plurality of segments, hereinafter called as the small blocks Bs, and extracts respective image characteristic values of the small blocks Bs from the cropped image data. The image characteristic values are numerical data representative of characteristics of the image data, such as color tint, color distribution, contour distribution, shape, spectral frequency distribution or components. In the present embodiment, image characteristic values representative of a blood vessel pattern in each of the small blocks Bs are extracted. Note that the small blocks Bs may correspond in size to the ranging blocks B or may have a smaller size than the ranging blocks B.

As exemplars of the image characteristic values representative of the blood vessel patterns, “direction distribution of vascular edges” and “magnitude distribution” are referable. “Direction distribution of vascular edges” represents the distribution of the directions in which edges of the blood vessels extend. More specifically, all blood vessels in the check zone C are segmentalized at constant intervals, and the distribution of the directions (0 to 180 degrees) of the blood vessel segments is detected as the direction distribution of vascular edges, wherein an appropriate direction is predetermined to be a referential direction (0 degree).

To detect “magnitude distribution”, four directional derivative filters of 3×3-pixel size are applied to each target pixel, to get the largest absolute value among output values from each of the four directional derivative filters, like as disclosed for example in JPA 09-138471, especially in FIG. 4 of this prior art. Then, distribution of the largest values is taken as the magnitude distribution. Moreover, it is possible to use magnitude distributions in the respective directions. It is also possible to use another known device, such as prewitt filters or Sobel filters, as the directional derivative filters.

Since the method of extracting image characteristic values of the blood vessel patterns is well known in the art, the description of this method will be omitted. After extracting the image characteristic values of the small blocks Bs from the cropped image data, the characteristic value extractor 82 writes these image characteristic values temporarily in the RAM 60.

The judgment section 83 reads out the image characteristic values of the small blocks Bs of the check zone C from the RAM 60, and compares these image characteristic values, to judge whether there is any area of concern 80 like a lesion in the check zone C. The image characteristic values of such small blocks Bs that correspond to the area of concern 80 differ greatly from the image characteristic values of other small blocks Bs that correspond to normal area 85. Therefore, if there is an area of concern 80 in the check zone C, some small blocks Bs in the check zone C have different image characteristic values from other small blocks Bs have. Namely, the check zone C includes a cluster of small blocks Bs that are less similar to other small blocks Bs if the area of concern 80 exits in the check zone C.

So the judgment section 83 compares the image characteristic values of the small blocks BS with each other, and judges that there is an area of concern 80 in the check zone C when there are a cluster of small blocks BS whose image characteristic values differ from ones of other small blocks BS to such an extent that is beyond a predetermined threshold value. For example, when some small blocks Bs have remarkably different image characteristic values from those of their neighboring small blocks BS, the judgment section 83 judges that an area of concern 80 exits in the check zone C. In that case, the judgment section 83 calculates differences between the image characteristic values of every couple of adjoining small blocks BS, and compares the calculated differences with respective threshold values. If all of the differences are less than the threshold values, the judgment section 83 judges that no area of concern 80 exits in the check zone C.

The judgment section 83 would get the same result when a lesion extends over the whole check zone C. Whether the lesion extends over the whole check zone C or not may be determined by referring to the image characteristic values of a previous image frame that is judged to have no lesion or area of concern 80. However, since the probability of occurrence of such a case is very low, the present embodiment is designed to judge that no area of concern 80 exists in the check zone C when none of the calculated differences reach or exceed the threshold values.

When some of the calculated differences reach or exceed the threshold values, the judgment section 83 judges that there is an area of concern 80 in the check zone C. In that case, a border between a couple of small blocks Bs, of which the differences between the image characteristic values reach or exceed the threshold value, is held as a border between the area of concern 80 and other normal area 85 that is not a lesion. That is, one small block BS of this couple belongs to the area of concern 80, while the other small block Bs of this couple belongs to the normal area 85.

When the differences in image characteristic values between a couple of adjacent small blocks Bs are less than the predetermined threshold values, the judgment section 83 regards that the two small blocks Bs belong to the same part or group. Accordingly, if any of the calculated differences between the adjacent small blocks Bs are not less than the threshold values, the check zone C is divided into at least two fractions: the area of concern 80 and the normal area 85. Then, the judgment section 83 determines which fraction of the check zone C is the area of concern 80. Namely, the judgment section 83 determines which one of the adjacent two small blocks Bs should belong to the area of concern 80 when the calculated difference between them reaches or exceeds the threshold value.

For example, since the area of concern 80 is rarely larger than the normal area 85, it is possible to determine the largest fraction to be the normal area 85, and other fractions to be the area of concern 80. Alternatively, it is possible to utilize the result of judgment on the check zone C of the previous image frame. Concretely, each time the judgment section 83 judges that no area of concern 80 exits in the check zone C, the judgment section 83 overwrites the RAM 60 with the image characteristic values of an arbitrary small block Bs in this check zone C. Because the surface condition and color of the inner wall of the body tract 10a little change within a limited area, the image characteristic values of the normal area 85 of the latest image frame little differs from the image characteristic values of written in the RAM 60. Therefore, among the two or more fractions, one having such image characteristic values that differ the least from the image characteristic values written in the RAM 60 is identified as the normal area 85, and other fraction or factions are determined to be the area of concern 80. This way, those small blocks Bs which constitute the area of concern 80 are discriminated from others. However, the method of discriminating the area of concern 80 is not limited to the present embodiment.

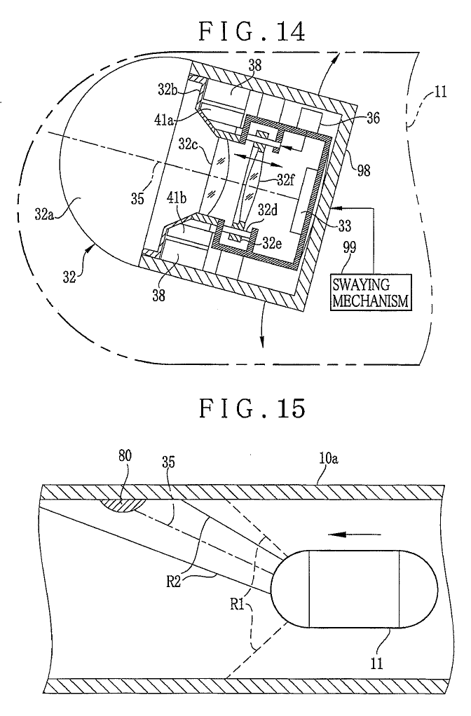

As described so far, the judgment section 83 judges whether there is any area of concern 80 in the check zone C of the latest or present image frame. If there is an area of concern 80 in the check zone C, the judgment section 83 distinguishes the small blocks Bs that constitute the area of concern 80 in the above-described manner. The result of judgment and discrimination by the judgment section 83 are fed to the CPU 57. The CPU 57 chooses the special imaging mode for the capsule endoscope 11 when the area of concern 80 exists in the check zone C, or chooses the regular imaging mode for the capsule endoscope 11 when any area of concern 80 does not exist in the check zone C. Note that the discrimination of those small blocks Bs which constitute the area of concern 80 is utilized in a third embodiment in a manner as set forth later with reference to FIG. 14, and is also usable to display a marker or the like indicating the area of concern 80 on the monitor 26 of the workstation 13.

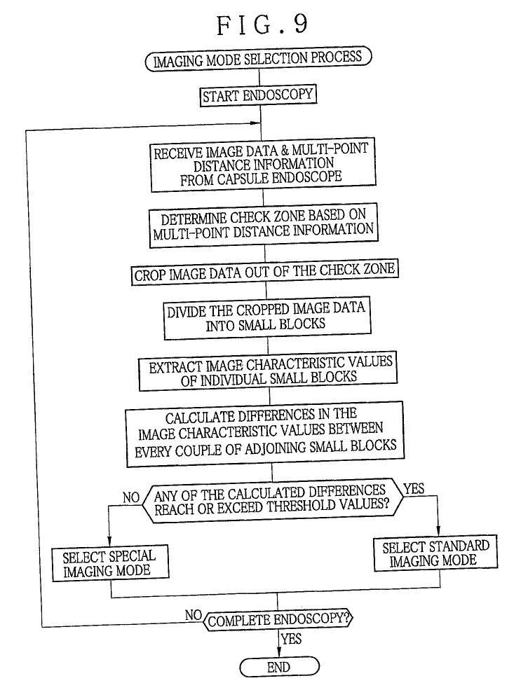

Next, a procedure for selecting the imaging mode of the capsule endoscope 11, which is executed in the data transceiver 12, will be described with reference to FIG. 9. When an endoscopy starts, the imaging device 33 of the capsule endoscope 11 captures an image from the subject in the field of view, i.e. the imaging field A, and the multi-point ranging sensor 41 makes the multi-point ranging of the imaging field A simultaneously. Image data of the captured image and the multi-point distance information on the imaging field A, from which the image data has been obtained, are sent on the radio wave 14a from the capsule endoscope 11 to the data transceiver 12.

The data transceiver 12 receives the radio wave 14a on the antennas 20, and transmits it via the receiver-transmitter circuit 75 to the demodulator circuit 62, to demodulate it into the original image data and the multi-point distance information. The image data is output to the image processing circuit 63, while the multi-point distance information is stored in the RAM 60. The image data is subjected to various image processing in the image processing circuit 63 and is, thereafter, output to the image analyzer circuit 67.

The cropping processor 81 of the image analyzer circuit 67 reads out the multi-point distance information from the RAM 60, corresponding to the image data as fed from the image processing circuit 63. On the basis of the read multi-point distance information, the cropping processor 81 determines the check zone C, and crops the image data to cut the check zone C out. The cropped image data is temporarily written in the RAM 60. The image characteristic value extractor 82 reads out the cropped image data from the RAM 60.

The characteristic value extractor 82 divides the check zone C, which corresponds to the cropped fragment of the image data, into a plurality of small blocks Bs, and extracts respective image characteristic values of the small blocks Bs from the cropped image data. The extracted image characteristic values of the small blocks Bs are temporarily written in the RAM 60. The judgment section 83 reads out the image characteristic values of the small blocks Bs from the RAM 60.

The judgment section 83 calculates differences in image characteristic values between every couple of adjoining small blocks Bs, and judges whether there is any area of concern 80 in the check zone C of the present image frame, depending upon whether any of the calculated differences reach or go beyond their threshold values. In other words, the judgment section makes the judgment as to whether any area of concern 80 exists in the check zone C, on the basis of a result of judgment as to whether there is a relative change in the image characteristic values in the check zone C, that is, whether there is such small blocks Bs that are less similar to other small blocks.

When the judgment section 83 judges that the check zone C of the present image frame contains an area of concern 80, the judgment section 83 distinguishes those small blocks Bs which constitute the area of concern 80 by means of the above-described method. The judgment section 83 outputs the result of judgment and data of the distinguished small blocks Bs to the CPU 57.

The CPU 57 chooses the special imaging mode for the capsule endoscope 11 when the judgment section 83 judges that the area of concern 80 exits in the check zone C of the present image frame. When the judgment section 83 judges that the area of concern 80 does not exit in the check zone C of the present image frame, the CPU 57 chooses the regular imaging mode for the capsule endoscope 11. In the same way as described above, the data transceiver 12 repeats the imaging mode selection processes each time it receives a new frame of image data from the capsule endoscope 11.

Note that the above imaging mode selection processes are repeated even after the capsule endoscope 11 is switched to the special imaging mode. But if an imaging field A of an endoscopic image (image data) obtained in the special imaging mode is narrower than the check zone, i.e. the distance range from D1 to D2 (see FIG. 7), the cropping process is skipped.

Referring back to FIG. 5, after selecting the imaging mode on the basis of the result of judgment by the judgment section, the CPU 57 decides the imaging conditions: the zooming magnification (field of view), the frame rate, the exposure value (the shutter speed and the illumination light volume), in accordance with the selected imaging mode, with reference to an imaging condition table 87 in the database 68.

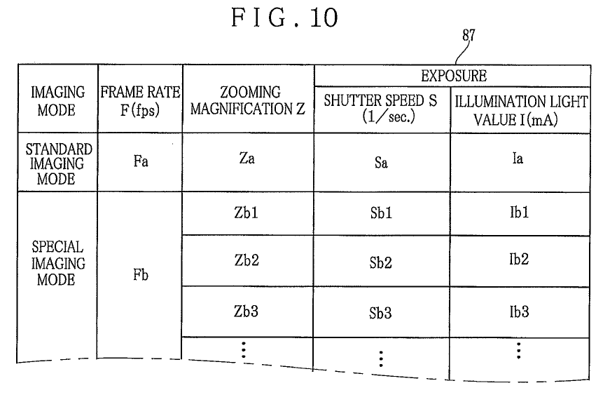

As shown in FIG. 10, the imaging condition table 87 defines the imaging conditions for the regular imaging mode and those for the special imaging mode, wherein plural sets of imaging conditions are prepared for the special imaging mode, because the capsule endoscope 11 captures images while varying the zooming magnification and the exposure value stepwise in the special imaging mode.

As the frame rate F (fps: frame per second), a higher value Fb is preset for the special imaging mode than a frame rate Fa preset for the regular imaging mode. So the capsule endoscope 11 will not fail to capture an image of the area of concern 80 even if the capsule endoscope 11 suddenly travels faster in the special imaging mode. The high frame rate Fb enables the capsule endoscope 11 to capture more endoscopic images of the area of concern 80 during a period from when the area of concern 80 enters the field of view of the objective lens system 32 till the area of concern 80 exits the field of view, so it is possible to capture the images of the area of concern 80 while varying the zooming magnification and the exposure value stepwise. The low frame rate Fa for the regular imaging mode reduces the power consumption by the capsule endoscope 11, and also reduces the number of endoscopic images captured from outside the area of concern 80, i.e. unnecessary images for diagnosis.

As the zooming magnification Z, a value Za for the regular imaging mode is preset on a wide-angle side, whereas values Zb1, Zb2, Zb3 . . . for the special imaging mode are preset on a telephoto side, so as to obtain enlarged images of the area of concern 80 in the special imaging mode. Moreover, in the special imaging mode, the zooming magnification is changed stepwise from the telephoto side toward the wide-angle side, or vise versa. Thus, at least an image of the area of concern 80 is captured at an optimum zooming magnification, i.e. at a maximum image magnification, wherever the area of concern 80 exists in the check zone C.

Since the capsule endoscope 11 captures images while moving through the tract 10a, there is a possibility that the area of concern 80 gets out of the field of view of the objective lens system 32 before the capsule endoscope 11 starts imaging in the special imaging mode. Therefore, at least one of the zooming magnification values Zb1, Zb3, Zb3 . . . for the special imaging mode may be set closer to a wide-angle terminal than the zooming magnification value Za for the regular imaging mode, so as to provide a wider field of view in the special imaging mode than in the regular imaging mode.

The exposure value is determined by the shutter speed S (1/sec.) and the illumination light volume I, which is controlled by a drive current (mA) supplied to the illuminator light source 38. The shutter speed S and the illumination light volume I are fixed in the regular imaging mode: S=Sa and I=Ia. On the other hand, in the special imaging mode, the shutter speed S and the illumination light volume I are gradually raised: S=Sb1, Sb2, Sb3 . . . , I=Ib1, Ib2, Ib3 . . . . Since the capsule endoscope 11 captures images while moving through the tract 10a, the condition of the illumination light incident on the subject, including the area of concern 80, varies with a change in attitude of the capsule endoscope 11. Capturing images while varying the exposure value (the shutter speed S and the illumination light volume I) stepwise will make the exposure condition of at least one of the captured images proper. In the regular imaging mode, the shutter speed S and the illumination light volume I are preferably set at lower level Sa and Ia than in the special imaging mode, because it reduces the power consumption by the capsule endoscope 11.

As shown in FIG. 5, the CPU 57 selects the imaging mode of the capsule endoscope 11 on the basis of the result of judgment by the judgment section and the imaging condition table 87, and decides the imaging conditions according to the selected imaging mode. Then the CPU 57 outputs a control command corresponding to the decided imaging conditions to the modulator circuit 61. The modulator circuit 61 modulates the control command into the radio wave 14b and outputs it via the receiver-transmitter circuit 75 to the antennas 20. Thus, the control command is sent wirelessly from the data transceiver 12 to the capsule endoscope 11.

As shown in FIG. 4, the radio wave 14b is received on the antenna 18 of the capsule endoscope 11, and is demodulated through the receiver-transmitter circuit 55 and the demodulator circuit 51 into the original control command. The control command is output to the CPU 45, so the zooming magnification (field of view), the frame rate, the exposure value (the shutter speed and the illumination light volume), which are designated by the control command, are temporarily written in the RAM 47.

The imaging driver 48 reads out the frame rate and the shutter speed from the RAM 47, and controls the imaging device 33 and the signal processing circuit 54 so that an endoscopic image is captured at the frame rate and the shutter speed as designated by the control command.

The lens driver 36 reads out the zooming magnification from the RAM 47, and adjusts the length of the objective lens system 32 by moving the second lens 32f so that the endoscopic image is captured at the zooming magnification as designated by the control command.

The illuminator driver 53 reads out the illumination light volume from the RAM 47, and controls the drive current applied to the illuminator light source 38 so that the endoscopic image is captured under the illumination light whose volume is designated by the control command. Thus, the capsule endoscope 11 captures the endoscopic image under the imaging conditions designated by the control command.

Moreover, in the special imaging mode, the imaging device 48, the lens driver 36 and the illuminator driver 53 control the imaging device 33, the signal processing circuit 54, the second lens 32f and the illuminator light source 38 respectively, so as to change the shutter speed, the zooming magnification and the illumination light volume stepwise.

A series of operations as described above: (1) image-capturing by the capsule endoscope 11, (2) sending of an endoscopic image to the data transceiver 12, (3) selecting the imaging mode, (4) generating the control command, (5) sending the control command to the capsule endoscope 11, and (6) controlling operation of the respective components of the capsule endoscope 11 on the basis of the control command, are cyclically repeated till an ending command is sent from the data transceiver 12 to the capsule endoscope 11 at the end of the endoscopy. These operations are performed speedy enough as compared to the speed of movement of the capsule endoscope 11. So the capsule endoscope 11 is switched to the special imaging mode as soon as the area of concern 80 is found in the regular imaging mode, before the area of concern 80 gets out of the field of view of the capsule endoscope 11.

As shown in FIG. 11, the overall operation of the workstation 13 is under the control of a CPU 90. The CPU 90 is connected via a data bus 91 to an LCD driver 92 for controlling the LCD 26, a communication interface (I/F) 94 for intermediating data-exchange through a USB connector 93 between the workstation 13 and the data transceiver 12, a data storage 95 and a RAM 96.

The data storage 95 stores image data that is taken out of the focused image data storage section 64b of the data transceiver 12. The data storage 95 also stores various programs and data necessary for the operation of the workstation 13, software programs for assisting doctors to make diagnoses, and diagnostic information sorted according the individual patients. The RAM 96 stores temporarily those data as read out from the data storage 95, and intermediate data as produced during various computing processes. When the assisting software is activated, a work window of the assisting software is displayed, for example, on the LCD 26. On this window, the doctor can display and edit some images or enter the diagnostic information by operating the operating section 25.

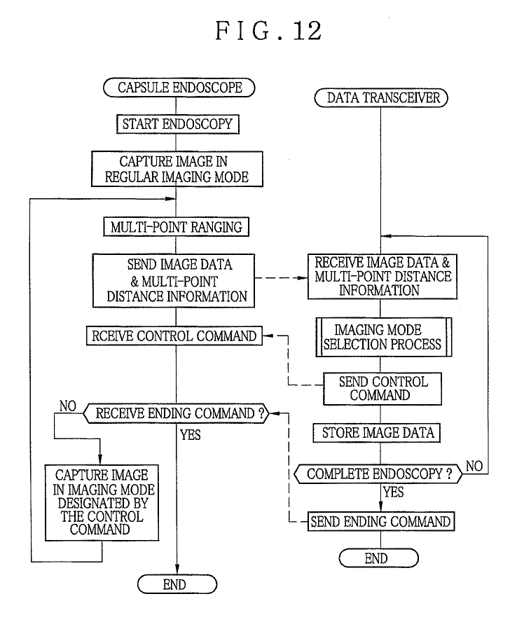

Now the operation of the capsule endoscopy system 2 as configured above will be described with reference to FIG. 12. Preliminary to an endoscopy, the doctor makes the patient 10 put on the shield shirt 19, the antennas 20 and the data transceiver 12, and turns the capsule endoscope 11 on.

When the patient 10 has swallowed the capsule endoscope 11 and gets ready for the endoscopy, the capsule endoscope 11 starts capturing images of the subject, i.e. the interior of the patient's tract, in the regular imaging mode. The illuminator light source 38 illuminates the subject, and an optical image of the subject is formed by the objective lens system 32 on the imaging surface of the imaging device 33, so the imaging device 33 outputs the analog image signal corresponding to the optical image. The image signal is fed to the signal processing circuit 54, and is converted to the digital image data through correlated double sampling, amplification, and analog-to-digital conversion. The image data is subjected to various image processing as described above with reference to FIG. 4.

With the start of the endoscopy, the multi-point ranging sensor 41 starts the multi-point ranging, wherein the multi-point ranging sensor 41 divides the imaging field A into the ranging blocks B of M×N matrix, and measures distances from the capsule endoscope 11 to the respective representative points P of the ranging blocks B. The multi-point ranging sensor 41 outputs the distance measuring signals to the distance signal converter circuit 49, which converts the distance measuring signals to the distance signals, and outputs them to the CPU 45. The CPU 45 outputs the distance signals of all the representative points P of the imaging field A as the multi-point distance information to the modulator circuit 50.

The digital image data output from the signal processing circuit 54 and the multi-point distance information from the CPU 45 are modulated into the radio wave 14a in the modulator circuit 50. The modulated radio wave 14a is amplified and band-pass filtered in the receiver-transmitter circuit 55 and is, thereafter, sent out from the antenna 18. Thus, the image data and the multi-point distance information on the imaging field A, from which the image data is obtained, are wirelessly sent from the capsule endoscope 11 to the data transceiver 12. At the same time, the electric field strength sensors 21, which are attached to the antennas 20, measure the strength of the electric field of the radio wave 14a from the capsule endoscope 11, and input the results of measurement to the position detector circuit 66 of the data transceiver 12.

The radio wave 14a is received on the antennas 20 of the data transceiver 12, and is fed through the receiver-transmitter circuit 75 to the demodulator circuit 62, which demodulates the radio wave 14a into the original image data and the multi-point distance information. The demodulated image data is subjected to various image processing in the image processor circuit 63, and is output to the image analyzer circuit 67 and the data storage 64. The demodulated multi-point distance information is temporarily written in the RAM 60.

The position detector circuit 66 detects the present position of the capsule endoscope 11 inside the patient 10 on the basis of the results of measurement of the electric field strength sensors 21, and outputs the detected present position as the imaging position data to the data storage 64. The data storage 64 records the imaging position data in association with the image data from the image processor circuit 63. The image data obtained in the regular imaging mode is stored in the ordinary image data storage section 64a. Note that the image data stored in the ordinary image data storage section 64a may be subjected to an appropriate data volume reduction process like a data compression process.

Each time the image analyzer circuit 67 is supplied with the image data from the image processing circuit 63, the image analyzer circuit 67 reads out the multi-point distance information corresponding to the image data from the RAM 60. Then, the image analyzer circuit 67, including the cropping processor 81, the image characteristic value extractor 82 and the judgment section 83, carry out the imaging mode selection processes as described above with reference to FIG. 9: (a) cropping image data of the check zone C, (b) extracting image characteristic values of the individual small blocks Bs, (c) judging whether there is any area of concern 80 in the check zone C, and, if there is one, (d) distinguishing those small blocks BS which constitute the area of concern 80.

The image analyzer circuit 67 outputs the result of judgment as to whether there is any area of concern 80, and if there is one, data of the distinguished small blocks Bs that constitute the area of concern 80. On the basis of the result of judgment by the image analyzer circuit 67, the CPU 57 selects the imaging mode of the capsule endoscope 11 and refers to the imaging condition table 87 of the database 68 to decide the imaging conditions according to the selected imaging mode, see FIG. 10. Then the CPU 57 generates a control command designating the decided imaging conditions and outputs the control command to the modulator circuit 61. The modulator circuit 61 modulates the control command into the radio wave 14b, and outputs it through the receiver-transmitter circuit 75 to the antennas 20. Thus the control command is wirelessly sent from the data transceiver 12 to the capsule endoscope 11.

The radio wave 14b is received on the antenna 18 of the capsule endoscope 11, and is demodulated into the original control command through the receiver-transmitter circuit 55 and the demodulator circuit 51. Then the control command is output to the CPU 45. As a result, the imaging conditions designated by the control command, i.e. a frame rate, a zooming magnification and an exposure value (a shutter speed and an illumination light volume), are temporarily written in the RAM 47.

The imaging driver 48 controls the imaging device 33 and the image processing circuit 54 so that endoscopic images are captured at the frame rate and the shutter speed as designated by the control command. The lens driver 36 controls the objective lens system 32 so that the endoscopic images are captured at the zooming magnification as designated by the control command. The illuminator driver 53 controls the drive current to the illuminator light source 38 so that the endoscopic images are captured at the illumination light volume as designated by the control command.

Consequently, if there is an area of concern 80 in the check zone C of an endoscopic image (image data frame) that is newly obtained in the regular imaging mode, the capsule endoscope 11 starts capturing images of the area of concern 80 in the special imaging mode. Concretely, the capsule endoscope 11 captures the images of the area of concern 80 at a higher frame rate while varying the zooming magnification and the exposure value stepwise. Thereby, at least one of the captured images will finely reproduce the area of concern 80. The image data obtained in the special imaging mode is stored in the focused image data storage section 64b of the data storage 64 of the data transceiver 12.

If there is no area of concern 80 in the check zone C, the capsule endoscope 11 continues image-capturing in the regular imaging mode. Since the frame rate, shutter speed and the illumination light volume are maintained in lower levels in the regular imaging mode as compared to the special imaging mode, power consumption by the capsule endoscope 11 is reduced.

When the judgment section 83 judges that the area of concern 80 does not exist in the check zone C of the latest image frame obtained in the special imaging mode, it means that the area of concern 80 gets out of the field of view of the capsule endoscope 11. Then, the data transceiver 12 generates a control command for resetting the capsule endoscope 11 to the regular imaging mode, and sends this control command wirelessly to the capsule endoscope 11. Thus, the capsule endoscope 11 is switched from the special imaging mode to the regular imaging mode.

Thereafter, the same operations as described above: (1) image-capturing by the capsule endoscope 11, (2) sending of an endoscopic image to the data transceiver 12, (3) selecting the imaging mode, (4) generating the control command, (5) sending the control command to the capsule endoscope 11, and (6) controlling operation of the respective components of the capsule endoscope 11 on the basis of the control command, are cyclically repeated till the ending command is sent from the data transceiver 12 to the capsule endoscope 11 at an end of the endoscopy.

To conclude the endoscopy, the data transceiver 12 is connected to the processor 24 through the USB cable 27, to transfer the image data from the focused image data storage section 64b of the data storage 64 of the data transceiver 12 to the processor 24. Then the doctor operates the operating section 25 to display the fine endoscopic images of the area of concern 80, which have been obtained in the special imaging mode, successively on the LCD 26, to interpret them.

As described so far, in the capsule endoscopy system 2 of the present embodiment, the check zone C of each endoscopic image frame obtained at present by the capsule endoscope 11 is divided into the small blocks Bs, and the image characteristic values extracted from one small block Bs are compared with those extracted from another small block BS, to check relative variations in the image characteristic values. Based on the relative variations, the capsule endoscopy system 2 makes the judgment as to whether there is any area of concern 80 in the check zone C. Therefore, the capsule endoscopy system 2 can determine the area of concern 80 exactly without any diagnostic information on past diagnoses of the patient or case information on general cases. Moreover, the capsule endoscopy system 2 can identify such a lesion that is not similar to a general image of the lesion shown in the case information. Because the capsule endoscopy system 2 does not need the diagnostic information or the case information, there is no need for considering the difference between the endoscope used for the present endoscopy and ones used for obtaining the diagnostic information or the case information.

Now a second embodiment of the present invention will be described, which differs from the above-described first embodiment in the way of making the judgment as to whether any area of concern exits in the check zone C or not.

Like the first embodiment, the second embodiment divides the check zone C of the present image frame into the small blocks Bs and extracts image characteristic values of the small blocks Bs from the cropped image data of the check zone C. However, in the second embodiment, the judgment as to whether any area of concern 80 exits in the check zone C or not is made based on the degree of similarity between the image characteristic values of the small blocks Bs of the latest or present image frame and ones of the small blocks Bs of the preceding image frame that has been obtained immediately before the present image frame. Because the second embodiment may have the same structure as the first embodiment and merely differs from the first embodiment in the way of analyzing the image data, the second embodiment will be described with reference to the same drawings as used for the first embodiment.

In a data transceiver 12 of the second embodiment, a RAM 60 or another memory device stores image characteristic values of the small blocks Bs of the present image frame as well as image characteristic values of the small blocks Bs of the preceding image frame, which are extracted by an image characteristic value extractor 82 of an image analyzer circuit 67. Each time the data transceiver 12 receives the image data newly from the capsule endoscope 11, the image characteristic values of the preceding image frame are replaced with those image characteristic values which are extracted from the new image data. Thus, the RAM 60 always stores two sets of image characteristic values of the respective check zones C of the latest and preceding image frames.

The image analyzer circuit 67 reads out the image characteristic values from the RAM 60, to calculate differences in the image characteristic values between each individual small block Bs of the present image frame and a corresponding small block Bs of the preceding image frame. For example, the corresponding small block Bs is one located in the same position (coordinative position) in the check zone C of the preceding image frame as the one small block Bs of the present image frame. The judgment section checks if any of the calculated differences reach or exceed the predetermined threshold values. Namely, the judgment section checks whether there are such small blocks Bs in the check zone C of the present image frame that have different image characteristic values from those the corresponding small blocks Bs of the preceding image frame have.

When none of the calculated differences reach or exceed the threshold values, the judgment section judges that an image fragment contained in the check zone C of the present image frame is similar to an image fragment contained in the check zone of the preceding image frame. Then, if the judgment section has judged that there is no area of concern 80 in the check zone C of the preceding image frame, the judgment section judges that no area of concern 80 exists in the check zone C of the present image frame. On the contrary, if the judgment section has judged that there is an area of concern 80 in the check zone C of the preceding image frame, the judgment section judges that the area of concern 80 exists in the check zone C of the present image frame too. This means that the area of concern 80 exists at the same position (small blocks Bs) in the check zone C of the present image frame as the position (small blocks Bs) in the check zone C of the preceding image frame. Such a result can be obtained for example while the capsule endoscope 11 stagnates in the tract 10a, or when a lesion (the area of concern 80) extends over a wide area of the tract 10a.

On the other hand, when some of the calculated differences reach or exceed the threshold values, the judgment section judges that the present image frame has some small blocks Bs whose image characteristic values change from those of the same small blocks Bs of the preceding image frame, and that an image fragment contained in the check zone C of the present image frame is not similar to an image fragment contained in the check zone of the preceding image frame. Then, if the judgment section has judged that there is no area of concern 80 in the check zone C of the preceding image frame, the judgment section judges that an area of concern 80 exists in the check zone C of the present image frame. In that case, those small blocks Bs having the changed image characteristic values are considered to constitute the area of concern 80. On the contrary, if the judgment section has judged that there is an area of concern 80 in the check zone C of the preceding image frame, it may probably be considered that the area of concern 80 does not exist in the present image frame, or there is a lesion or an area of concern 80 in the present image frame but it exists in a different position or has a different contour from the area of concern 80 of the preceding frame. Therefore, in that case, it is preferable to check whether any area of concern exists in the check zone C of the present frame on the basis of similarity in the image characteristic values between the small blocks Bs of the present frame, in the same way as described with respect to the first embodiment.

It is to be noted that the method of judgment according to the second embodiment is usable only when the present image frame is obtained in the same imaging mode as the preceding image frame. If the present image frame is obtained in the regular imaging mode while the preceding image frame was obtained in the special imaging mode, or in the opposite case, the quality of the present image frame differs from that of the preceding image frame due to the differences in zooming magnification and exposure value. Therefore, it is hard to distinguish the area of concern 80 by comparing the present and preceding image frames, which are obtained in the different imaging modes from each other. In that case, the judgment as to whether any area of concern 80 exists should be made according the method of the first embodiment.

The result of judgment by the judgment section is fed to the CPU 57. The CPU 57 selects the imaging mode of the capsule endoscope 11 depending upon whether any area of concern 80 exits in the check zone C of the present image frame or not, in the manner as described above.

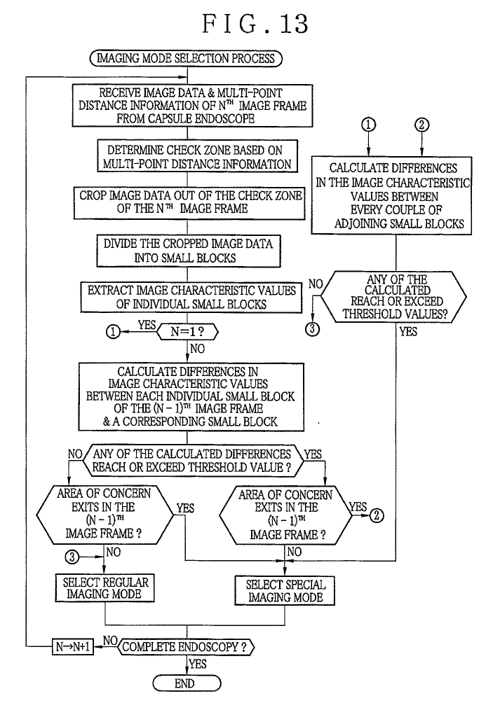

Next, the imaging mode selection processes of the second embodiment will be described with reference to FIG. 13. When an endoscopy starts, the capsule endoscope 11 stars capturing images of the object in the regular imaging mode and also starts the multi-point ranging. Thus, the capsule endoscope 11 wirelessly sends out image data of the captured images and the multi-point distance information sequentially to the data transceiver 12. When the data transceiver 12 receives the image data of a first image frame, a cropping processor 81 crops image data pieces out of the check zone C, and the image characteristic extractor 82 extracts respective image characteristic values of the small blocks Bs, in the manner as described in the first embodiment. The extracted image characteristic values of the small blocks Bs of the first image frame are written in the RAM 60.

As for the first or initial image frame, the judgment as to whether there is any area of concern 80 in the check zone C is preferably made according to the method of the first embodiment. Note that the following description is based on the assumption that no area of concern 80 exits in the check zone C of the first image frame.

After the image characteristic values of all small blocks Bs of the check zone C of a second or next image frame are written in the RAM 60, the judgment section reads out the image characteristic values of the second and first image frames from the RAM 60, to calculate differences in the image characteristic values between each individual small block Bs of the second or present image frame and the corresponding small block Bs of the first or preceding image frame.

When none of the calculated differences reach or exceed the threshold values, the judgment section judges that an image fragment contained in the check zone C of the present or second image frame is similar to an image fragment contained in the check zone of the preceding or first image frame. Since there is no area of concern 80 in the check zone C of the first image frame, the judgment section judges that no area of concern 80 exists in the check zone C of the second image frame.

On the other hand, when some of the calculated differences reach or exceed the threshold values, the judgment section judges that the second image frame has some small blocks Bs whose image characteristic values change from those of the same small blocks Bs of the first image frame. Then, since there is no area of concern 80 in the check zone C of the first image frame, the judgment section judges that an area of concern 80 exists in the check zone C of the second image frame, and distinguishes those small blocks Bs which have the changed image characteristic values and thus constitute the area of concern 80.

The judgment section outputs the result of judgment and the data of the distinguished small blocks Bs to the CPU 57. The CPU 57 selects the imaging mode of the capsule endoscope 11 depending upon whether any area of concern 80 exits in the check zone C of the second image frame.

As for the following image frames, each time the data transceiver 12 receives the image data of a new image frame from the capsule endoscope 11, the judgment section calculates differences in image characteristic values between each individual small block Bs of the N^{th} or present image frame and the corresponding small block Bs of the (N−1)^{th} or preceding image frame, and judges the presence or absence of an area of concern 80 in the check zone C on the basis of the calculated differences in the same way as described above.

As described above, if some of the calculated differences reach or exceed the threshold values after it is judged that an area of concern 80 exists in the check zone C of the preceding or (N−1)^{th} image frame, the judgment section 83 makes the judgment as to whether any area of concern 80 exits in the check zone C of the present or N^{th} image frame according to the method of the first embodiment. Also when the N^{th} image frame and the (N−1)^{th} image frame have been obtained in the different imaging modes from each other, the judgment as to whether any area of concern 80 exits in the check zone C of the N^{th} image frame is made according to the method of the first embodiment.

As described so far, according to the second embodiment, the judgment as to whether any area of concern 80 exits in the check zone C of the present image frame is made by comparing the present image frame with the preceding image about whether there are any small blocks Bs in the present image frame, whose image characteristic values change from ones the corresponding small blocks Bs have in the preceding image frame. Therefore, the second embodiment achieves the same effect as described with respect to the first embodiment.

Next, a third embodiment of the present invention will be described. Although the first and second embodiments have been described on the presumption that the optical axis 35 of the objective lens system 32 of the capsule endoscope 11 is fixed in a direction parallel to a lengthwise direction of the capsule endoscope 11, the present invention is not limited to this configuration. According to the third embodiment, the optical axis 35 of the objective lens system 32 is directed toward an area of concern 80 when the area of concern 80 is detected and the capsule endoscope 11 is switched to the special imaging mode.

In order to change the direction of the optical axis 35, as shown for example in FIG. 14, the capsule endoscope 11 is provided with a container 98 containing the objective lens system 32, the imaging device 33, the illuminator light source 38, the multi-point ranging sensor 41 and other necessary components for imaging, and a swaying mechanism 99 for the container 98. The swaying mechanism 99 sways the container 98 so as to incline the optical axis 35 of the objective lens system 32 in an appropriate direction. As well known in the art, the description of the swaying mechanism 99 will be omitted.

As described with reference to FIG. 8, when it is judged that an area of concern 80 exists in the check zone C, the small blocks Bs constituting the area of concern 80 are distinguished. Then, it is determined how much and in what direction the area of concern 80 deviates from the center of the check zone C, i.e. the center of the imaging field A, which is on the optical axis 35 of the objective lens system 32 in the regular imaging mode.

The direction and amount of the deviation of the area of concern 80 from the center of the check zone C are determined, for example, by the image analyzer circuit 67. As the deviation amount, the number of small blocks Bs or blocks B from the center to the area of concern 80 may be detected. Based on the deviation amount, the image analyzer circuit 67 determines an inclination angle of the optical axis 35 from the position in the regular imaging mode toward the area of concern 80. The inclination angle of the optical axis 35 according to the deviation amount of the area of concern 80 may be predetermined by measurement.

When the direction and angle of inclination of the optical axis 35 are determined, the CPU 57 of the data transceiver 12 generates a control command on the basis of the direction and angle of inclination of the optical axis 35, hereinafter referred to as optical axis adjustment information, and the imaging conditions as determined in the manner as described with respect to the first embodiment. The control command is sent wirelessly to the capsule endoscope 11, so the CPU 45 of the capsule endoscope 11 controls the swaying mechanism 99 to sway the container 98 to incline the optical axis 35 of the objective lens system 32 according to the optical axis adjustment information received as the control command. Thereby, the objective lens system 32 is directed toward the area of concern 80.

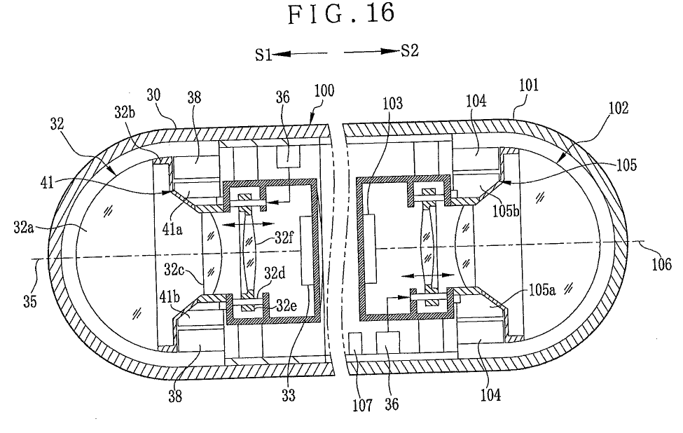

As shown in FIG. 15, the objective lens system 32 gets a pretty narrow field of view R2, as shown by solid line, by directing the optical axis 35 of the objective lens system 32 toward the area of concern 80 in the special imaging mode, in comparison with a field of view R1 in the regular imaging mode, as shown by dashed line. Therefore, in the special imaging mode, the capsule endoscope 11 will capture images while focusing on the area of concern 80. As a result, it becomes possible to obtain more enlarged and thus detailed endoscopic images of the area of concern 80 than those obtainable in the first embodiment.

Next a fourth embodiment of the present invention will be described. Although the capsule endoscope 11 used in the first and second embodiments has the objective lens system 32, the imaging device 33, the illuminator light source 38 and the multi-point ranging sensor 41 only on the side of the front casing 30, the present invention is not limited to these embodiments. For example, as shown in FIG. 16, a capsule endoscope 100 may have an objective lens system 102, an imaging device 103, an illuminator light source 104 and a multi-point ranging sensor 105 consisting of a photo emitter unit 105a and a photo sensor unit 105b on the side of its rear casing 101, beside an objective lens system 32, an imaging device 33, an illuminator light source 38 and a multi-point ranging sensor 41 on the side of its front casing 30. Needless to say, both the front and rear casings 30 and 101 are transparent. The objective lens systems 32 and 102, the imaging devices 33 and 103, the illuminator light sources 38 and 104 and the multi-point ranging sensors 41 and 105 respectively have the same structures as described in the first embodiment. Therefore, the description of these elements will be omitted. Designated by 106 is an optical axis of the objective lens system 102.

With the imaging devices 33 and 103 on the opposite sides, the capsule endoscope 100 captures images through one of these imaging devices 33 and 103: one facing forward in the traveling direction of the capsule endoscope 100 is used in the regular imaging mode, whereas the other facing backward in the traveling direction of the capsule endoscope 100 is used in the special imaging mode.

The following description is based on the assumption that the capsule endoscope 100 travels through a tract 10a in a direction substantially parallel to the optical axes 35 and 106, and that the capsule endoscope 100 can move with its front casing 30 forward or with its rear casing 101 forward. Whether the capsule endoscope 100 is moving with it front casing 30 forward or with its rear casing 101 forward is detected by a traveling direction detector or attitude sensor 107 that is built in the capsule endoscope 100. The traveling direction detector 107 is for example a uniaxial accelerometer. The detection result by the traveling direction detector 107, hereinafter referred to as traveling direction data, is seriatim sent together with the image data and the multi-point distance information to a data transceiver 12. Hereinafter, we will explain that the capsule endoscope 100 travels in a first direction S1 as it heads its front casing 30 forward, and that the capsule endoscope 100 travels in a second direction S2 as it heads its rear casing 101 forward, as implied by arrows in FIG. 16.

Instead of the traveling direction detector 107, it is possible to detecting the traveling direction of the capsule endoscope 100 on the basis of a variation in endoscopic images with time, which are successively obtained by the imaging device 33 or 103. As well known in the art, the process of detecting the traveling direction of the capsule endoscope will be omitted.

On the basis of the traveling direction data from the traveling direction detector 107, a CPU 57 of the data transceiver 12 (see FIG. 5) generates a control command for driving the imaging device 33 to capture images of the subject in the regular imaging mode, while the capsule endoscope 100 is traveling in the first direction S1. On the other hand, while the capsule endoscope 100 is traveling in the second direction S2, the CPU 57 of the data transceiver 12 (see FIG. 5) generates a control command for driving the imaging device 103 to capture images of the subject in the regular imaging mode. Simultaneously, the CPU 57 generates another control command for interrupting imaging of the imaging device that is located rearward in the traveling direction of the capsule endoscope 100. The control commands generated by the CPU 57 are sent wirelessly to the capsule endoscope 100. So the forward imaging device in the traveling direction captures images of the subject in the regular imaging mode, while the rearward imaging device stops imaging.

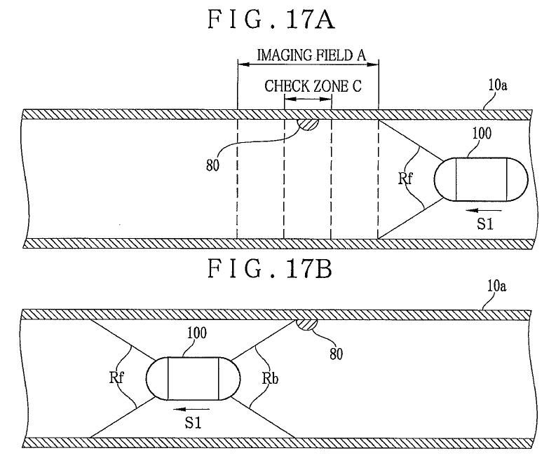

Accordingly, as shown in FIG. 17A, while the capsule endoscope 100 is traveling in the first direction S1, the imaging device 33 captures images of the subject in the regular imaging mode, and the image data, the multi-point distance information and the traveling direction data are sent wirelessly from the capsule endoscope 100 to the data transceiver 12. In the same way as described with respect to the above embodiments, the image analyzer circuit 67 of the data transceiver 12 judges whether any area of concern 80 exists in the check zone C of the imaging field A of the present image frame. Note that Rf and Rb represent respective fields of view of the objective lens system 32 and the objective lens system 102.

After the image analyzer circuit 67 judges that the area of concern 80 exists in the check zone C, the CPU 57 of the data transceiver 12 judges whether the area of concern 80 enters the field of view Rb of the objective lens system 102, as shown in FIG. 17B. The judgment as to whether the area of concern 80 enters the field of view Rb of the objective lens system 102 may be made by means of any appropriate method.

For example, on the basis of the multi-point distance information as obtained by the multi-point ranging, which has been sent to the data transceiver 12 together with the image data, the CPU 57 measures a distance “d” between the capsule endoscope 100 and the area of concern 80 at the moment when the present image frame was obtained. Thereafter when the capsule endoscope 100 travels the distance “d” in the direction S1, the capsule endoscope 100 comes into a range around the area of concern 80. Thereafter, when the capsule endoscope 100 travels farther a given distance “Δd” in the direction S1, the area of concern 80 enters the field of view Rb of the objective lens system 102, wherein “Δd” is longer than a whole length of the capsule endoscope 100 and varies depending upon the individual capsule endoscopes, so the distance “Δd” may be predetermined by measurement. In conclusion, the area of concern 80 will enter the field of view Rb of the objective lens system 102 when the capsule endoscope 100 travels a distance “d+Δd” in the direction S1 since the judgment that the area of concern 80 exits in the image frame obtained by the imaging device 33.

In a case where the capsule endoscope 100 uses an accelerometer as the traveling direction detector 107, information on the acceleration of the capsule endoscope 100 is wirelessly sent to the data transceiver 12. The CPU 57 of the data transceiver 12 calculates a travel distance of the capsule endoscope 100 on the basis of the acceleration information obtained by the accelerometer of the capsule endoscope 100, to judge that the area of concern 80 comes in the field of view Rb of the objective lens system 102 when the capsule endoscope 100 has moved by the distance “d+Δd” from the time when the area of concern 80 was found in the field of view of the imaging device 33.

Alternatively, it is possible to start driving the imaging device 103 in the regular imaging mode when it is judged that the area of concern 80 exists in the check zone C of the present imaging field A of the imaging device 33, and analyze each image frame obtained by the imaging device 103 in the image analyzer circuit 67 so as to detect whether the area of concern 80 exists in the check zone C of the image frame in the same manner as described above. When the area of concern 80 is found in the check zone C, the CPU 57 judges that the area of concern 80 enters the field of view Rb of the objective lens system 102.

When it is judged that the area of concern 80 enters the field of view Rb of the objective lens system 102, the CPU 57 of the data transceiver 12 generates a control command for driving the imaging device 103 to capture images of the area of concern 80 in the special imaging mode. The imaging conditions in the special imaging mode are decided in the same way as in the first embodiment. The control command is wirelessly sent from the data transceiver 12 to the capsule endoscope 100. Thus, the imaging device 103 captures images of the area of concern 80 in the special imaging mode.

While the capsule endoscope 100 is traveling in the second direction S2, the same operations as described above are carried out in the fourth embodiment, except but the imaging device 33 (the objective lens system 32) and the imaging device 103 (the objective lens system 102) exchange the roles with each other, so the description of this case will be omitted.

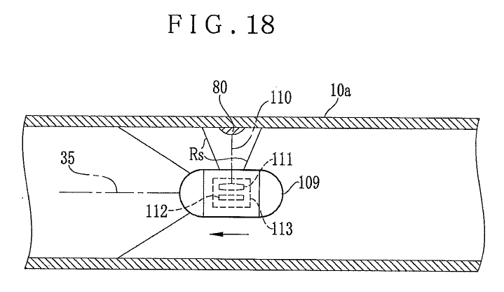

Although the fourth embodiment has been described in connection with the capsule endoscope 100 that has the imaging devices 33 and 103 on the front and rear sides in the casings 30 and 101, the present invention is also applicable to such a capsule endoscope 109 that can capture an optical image of the subject in a lateral direction of the capsule endoscope 109, as shown in FIG. 18. The capsule endoscope 109 has an imaging unit 113 mounted in its middle position, which is constituted of an objective lens system 111 whose optical axis 110 is perpendicular to a lengthwise direction of the endoscope 109, and an imaging device 112 placed behind the objective lens system 111. Like the above embodiments, the capsule endoscope 109 also has an objective lens system 32 and an imaging device 33 on its front side, though they are not shown in FIG. 18. The objective lens system 32 has an optical axis 35 that coincides with the lengthwise axis of the capsule endoscope 109.

Although it is not shown in the drawings, the capsule endoscope 109 is provided with a turning mechanism for turning the imaging unit 113 about the lengthwise axis of the capsule endoscope 109. Thereby, the optical axis 110 of the objective lens system 111 can rotate through 360 degrees around the optical axis 35. The objective lens system 111 and the imaging device 112 have the same structure as the objective lens system 32 and the imaging device 33.

The capsule endoscope 109 is controlled to capture images of the subject through the imaging device 33 in the regular imaging mode, and the judgment as to whether any area of concern 80 exits in a check zone C of the present image frame is carried out in a data transceiver 12. When it is judged that an area of concern 80 exits in the check zone C, a CPU 57 of the data transceiver 12 starts checking if the area of concern 80 comes in a field of view Rs of the objective lens system 111, in the same manner as described with respect to the fourth embodiment.

When the CPU 57 judges that the area of concern 80 comes in the field of view Rs of the objective lens system 111, the CPU 57 generates a control command for causing the optical axis 110 of the objective lens system 111 to turn in a direction toward the area of concern 80, and a control command for driving the imaging device 112 to capture images in a special imaging mode. These control commands are sent wirelessly from the data transceiver 12 to the capsule endoscope 109, so the imaging unit 113 is turned about the lengthwise axis of the capsule endoscope 109 to direct the optical axis 110 toward the area of concern 80 and thereafter the data transceiver 12 captures images of the area of concern 80 in the special imaging mode. Instead of turning the imaging unit 113 (the optical axis 110) about the lengthwise axis of the capsule endoscope 109 (the optical axis 35), it is possible to use a panorama lens for the objective lens system 111, which has an angle of view of 360 degrees.

It is also possible to use a capsule endoscope that is provided with an objective lens system 111 and an imaging device 112 like the capsule endoscope 109 of FIG. 18, as well as objective lens systems 32 and 102 and imaging devices 33 and 103 like the capsule endoscope 100 of FIG. 16. Namely, the capsule endoscope having three imaging devices respectively viewing front, side and rear of the traveling direction of the capsule endoscope is usable in such a manner that the front-viewing imaging device is driven in the regular imaging mode, while the other imaging devices are driven in the special imaging mode to capture images of an area of concern.

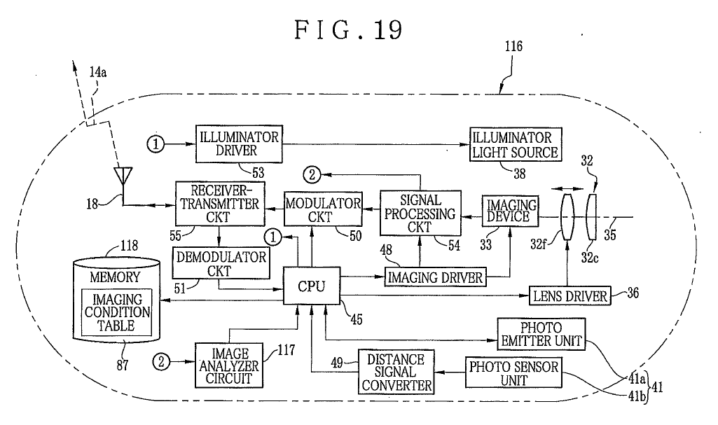

In the above described embodiment, the data transceiver 12 carries out the image analysis or the judgment as to whether there is any area of concern in the check zone of the endoscopic image and generates the control commands. However, the present invention is not limited to these embodiments, but the image analysis and the generation of the control commands may be carried out within a capsule endoscope. FIG. 19 shows an example of such capsule endoscope 116 that makes the image analysis and generates the control commands.

The 116 fundamentally has the same structure as the capsule endoscope 11 of the first embodiment, but the 116 is provided with an image analyzer circuit 117 and a memory 118. The 117 and the memory 118 take the same functions as the image analyzer circuit 67 and the database 68 of the data transceiver 12 of the first embodiment, respectively. The 117 has the same imaging condition table 87 as the database 68 has. Note that the 116 is provided with the same members as the 11 has, although some of them such as a ROM 46, a RAM 47 and a power supply circuit 52 are omitted from FIG. 19.

The 117 is fed with image data from a signal processing circuit 54, and multi-point distance data from a CPU 45. The 117 executes the imaging mode selection processes as described above with respect to the first and second embodiments: (a) cropping image data of the check zone C, (b) extracting respective image characteristic values of the small blocks Bs, (c) judging whether there is any area of concern 80 in the check zone C, and, if there is one, (d) distinguishing those small blocks Bs which constitute the area of concern 80.

The 117 outputs the result of judgment to a CPU 45. On the basis of the result of judgment by the image analyzer circuit 117, the CPU 45 selects the imaging mode of the capsule endoscope 116 and refers to the imaging condition table 87 of the memory 118 to decide the imaging conditions according to the selected imaging mode. Then the CPU 45 generates a control command designating the decided imaging conditions and controls the respective components of the 116 according to the control command. In this embodiment, the 116 sends out a radio wave 14a to an external apparatus, like a data transceiver, but does not receive a radio wave 14b from the external apparatus.

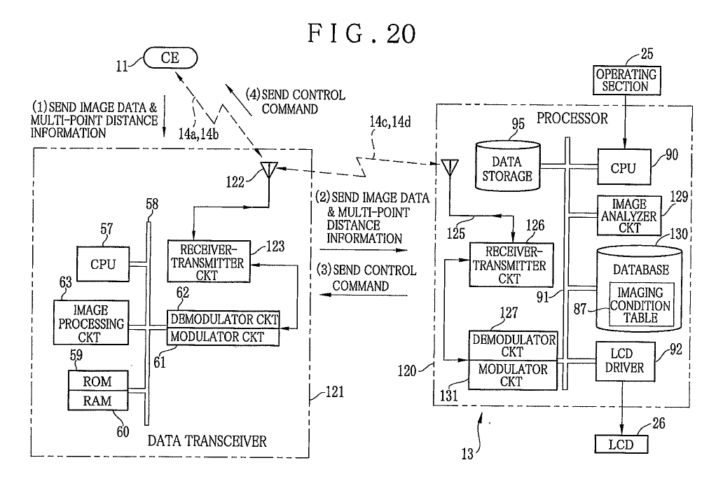

It is also possible to configure a workstation 13 such that a processor 120 of the workstation 13 makes the image analysis and generates the control commands in place of the data transceiver 12 of the first embodiment or the 116. In this embodiment, as shown in FIG. 20, a data transceiver 121 is wirelessly connected to the processor 120 of the workstation 13. Then, (1) a capsule endoscope 11 sends the image data and the multi-point distance information to the 121 on a radio wave 14a, and (2) the image data and the multi-point distance information are sent from the 121 to the 120 on a radio wave 14c. So the 120 analyzes the image data and generates the control commands, and (3) the control commands are sent from the workstation 13 to the 121 on a radio wave 14d, and then (4) the control commands are sent from the 121 to the 11 on a radio wave 14b.

Namely, the 121 relays or translates the image data and the multi-point distance information from the 11 to the 13, and also relays the control commands from the 13 to the 11. For this purpose, the 121 is provided with an antenna 122 and a receiver-transmitter circuit 123, which are capable of multi-data-communication. The radio wave 14a from the 11 is received on the 122 and is fed through the 123 to a demodulator circuit 62, to be demodulated into the original image data and the multi-point distance information. After the image data is processed in an image processing circuit 63, the processed image data and the multi-point distance information are modulated into the radio wave 14c in a modulator circuit 61. Note that the unprocessed image data may be modulated into the radio wave 14c. The radio wave 14c is fed through the 123 to the 122, so the image data and the multi-point distance information are wirelessly sent from the 121 to the 120.

The radio wave 14d as received on the 122 from the 120 is fed through the 123 to the demodulator circuit 62. Directly after the demodulator circuit 62 demodulates the radio wave 14d into the original control command, the modulator circuit 61 modulates the control command into the radio wave 14b. The radio wave 14b is output through the 123 to the 122, so the control command is wirelessly sent from the 121 to the 11.

The 120 exchanges data, including the image data, the multi-point distance information and the control commands, with the 121 by way of an antenna 125. The 120 is provided with a receiver-transmitter circuit 126, a demodulator circuit 127, an image analyzer circuit 129, a database 130 and a modulator circuit 131, beside those components which are described above with reference to FIG. 11, including a CPU 90, an LCD driver 92, and a data storage 95.

The 129 has the same function as the image analyzer circuit 67 of the first embodiment. The database 130 corresponds to the database 68 of the 12 of the first embodiment, and stores an imaging condition table 87. When the image data and the multi-point distance information is fed from the demodulator circuit 127, the 129 executes the imaging mode selection processes as described above with respect to the image analyzer circuit 67.

The result of judgment and other data obtained by the 129 are output to the CPU 90. On the basis of the result of judgment, the CPU 90 generates a control command with reference to the imaging condition table 87 of the database 130, and outputs the control command to the modulator circuit 131.

The modulator circuit 131 modulates the control command into the radio wave 14d and outputs the radio wave 14d through the 126 to the antenna 125, so the radio wave 14d representative of the control command is sent from the 120 to the 121.

The control command is wirelessly sent via the 121 to the 11 in the manner as described above. Then the 11 captures images in the imaging mode designated by the control command. Providing the 13 with the function to execute the image analysis and the control command generation contributes to making the data transceiver 121 compact and minimizing the capsule endoscope.

Although the above described embodiments execute the image analysis and the control command generation in one of the data transceiver, the capsule endoscope and the processor, these embodiments are not limiting the present invention. It may be possible to provide all of the data transceiver, the capsule endoscope and the processor with the function for executing the image analysis and the control command generation, so that one of them is selected to execute this function.

In the first embodiment, the judgment as to whether any area of concern 80 exits in the check zone C of the present image frame is made on the basis of similarity between the individual small blocks Bs of the check zone C, which is detected by comparing image characteristic values of the adjoining small blocks Bs. On the other hand, in the second embodiment, the judgment as to whether any area of concern 80 exits in the check zone C of the present image frame is made on the basis of similarity between the check zone C of the present image frame and the check zone C of the preceding image frame, which is detected by comparing image characteristic values of each individual small block Bs of the present image frame with those of the corresponding small blocks Bs of the preceding image frame. It is possible to execute the judgment process of the first embodiment and the judgment process of the second embodiment continually and simultaneously. Thereby the judgment about the presence of the area of concern 80 would be more precise.

Although the above described embodiments vary the zooming magnification and the exposure value stepwise in the special imaging mode for capturing successive images of the area of concern 80, the present invention is not limited to these embodiments, but it is possible to vary other factors of the imaging conditions stepwise. For example, it is possible to vary the focusing position or the kind or the number of the illuminator light sources. Then, the imaging device may capture images of the area of concern 80 at least once under proper focusing condition or proper lighting condition. Thus, high quality endoscopic images of the area of concern 80 would be obtained.

In the first embodiment makes the judgment about the presence of those small blocks Bs whose image characteristic values vary relatively largely from ones of other small blocks Bs by comparing differences in the image characteristic values between every couple of adjoining small blocks Bs with the threshold values. However, the present invention is not limited to this method, but any other similarity judging methods are usable to judge the presence of the small blocks Bs having different image characteristic values from others. For example, it is possible to calculate a degree of similarity in the image characteristic values between adjoining small blocks Bs by calculating a square sum of the differences, and compare the similarity degree with a predetermined threshold value. The same applies to the second embodiment.

Although the second embodiment makes the judgment as to whether there is any area of concern 80 in the check zone C of the present image frame on the basis of the similarity in the image characteristic values between the corresponding small blocks Bs of the respective check zones C of the present and preceding image frames, the present invention is not limited to this method. It is alternatively possible to calculate a degree of similarity between the respective check zones of the present and preceding image frames based on image characteristic values extracted from the cropped image data of the present image frame and ones of the preceding image frame. It is also possible to calculate a degree of similarity between the present and preceding image frames based on image characteristic values extracted respectively from the image data of the present and preceding image frames.

Although the judgment as to whether any area of concern 80 exits or not is made concerning the check zone C of the present image frame in the first and second embodiments, it is possible to check the presence of area of concern 80 across the whole imaging field A of the present image frame.

Although the illustrated capsule endoscopes change the zooming magnification by varying the focal length of the optical lens system, it is possible to vary the zooming magnification electronically. Where the zooming magnification is electronically varied, it is possible to make the field of view of the capsule endoscope variable by varying the magnification of the endoscopic image electronically through processing an image signal obtained by the imaging device 33.

In the above described embodiments, the capsule endoscope is switched to the special imaging mode only when it is judged that an area of concern 80 exits in the check zone C of the present image frame. However, it is possible to switch the capsule endoscope to the special imaging mode at predetermined intervals, i.e. periodically or at every traveling distance.

Although the multi-point ranging of the imaging field A, see FIG. 3, is carried out by the active multi-point ranging sensor 41 in the above embodiments, the present invention is not limited to this method. For example, such a multi-focus multi-point ranging method as known from JPA 2003-037767 is applicable, wherein an optimum focus position to each individual ranging block B of the imaging field A is detected while changing the position of a focus lens from far to near, and estimate distances to the respective ranging blocks B by the detected focus positions. It is also possible to apply such a stereo-type multi-point ranging method as known from JPA 2007-151826 or JPA 2006-093860 to a capsule endoscope, wherein the capsule endoscope is provided with at least two sets of objective lens systems having parallel optical axes and imaging devices disposed behind the respective lens systems, so that distances to the respective ranging blocks B are detected based on parallaxes between images captured simultaneously by these imaging devices.

Thus, the present invention is not to be limited to the above embodiments but, on the contrary, various modifications will be possible without departing from the scope of claims appended hereto.

What is claimed is:

1. A capsule endoscopy system comprising:
a capsule endoscope to be swallowed by a test body, said capsule endoscope comprising an imaging device to capture endoscopic images of internal portions of the test body and a sender for sending the endoscopic images wirelessly;
a portable apparatus that the test body may carry about, said portable apparatus comprising a receiver for receiving the endoscopic images from said capsule endoscope and a data storage for storing the received endoscopic images;
an information managing apparatus for storing and managing the endoscopic images that are transferred from said portable apparatus; and
a judging device that analyzes each endoscopic image immediately after it is obtained by said imaging device, to judge by a result of the analysis whether said endoscopic image contains any area of concern that has different image characteristics from surrounding areas, said judging device being mounted at least in one of said capsule endoscope, said portable apparatus and said information managing apparatus.

2. A capsule endoscopy system as recited in claim 1, further comprising:
a control command generator for generating control commands for controlling operations of respective members of said capsule endoscope on the basis of a result of the judgment by said judging device, said control command generator being mounted at least in one of said capsule endoscope, said portable apparatus and said information managing apparatus; and
an operation controller mounted in said capsule endoscope, for controlling operations of the respective members of said capsule endoscope in accordance with said control commands.

3. A capsule endoscopy system as recited in claim 1, wherein said judging device divides each endoscopic image into a plurality of segments, examines similarity among these segments, and judges that an area of concern exists in said endoscopic image when there are some segments that bear relatively low similarities to other segments of said endoscopic image.

4. A capsule endoscopy system as recited in claim 3, wherein said judging device detects image characteristic values from the respective segments, calculates differences in the image characteristic values between the respective segments, and estimates the similarity between the segments by comparing the calculated differences with predetermined threshold values.

5. A capsule endoscopy system as recited in claim 1, wherein said judging device examines similarity between the latest endoscopic image obtained from said capsule endoscope and the preceding image obtained immediately before from said capsule endoscope, and judges that an area of concern exists in the latest endoscopic image if the latest endoscopic image is not similar to the preceding image and said judging device has judged that no area of concern exits in the preceding image, or if the latest endoscopic image is similar to the preceding image and said judging device has judged an area of concern exits in the preceding image.

6. A capsule endoscopy system as recited in claim 5, wherein said judging device divides each endoscopic image into a plurality of segments, estimates similarity between the segments of the latest endoscopic image and corresponding segments of the preceding image, and judges that the latest endoscopic image is not similar to the preceding image when there are some segments that bear relatively low similarities to the corresponding segments of the preceding image.

7. A capsule endoscopy system as recited in claim 6, wherein said judging device detects image characteristic values from the respective segments of the latest and preceding endoscopic images, calculates differences in the image characteristic values between each individual segment of the latest endoscopic image and the corresponding segment of the preceding image, and estimates the similarity between each couple of the corresponding segments of the latest and preceding images by comparing the calculated differences with predetermined threshold values.

8. A capsule endoscopy system as recited in claim 1, wherein said capsule endoscope comprises a multi-point ranging device for measuring distances from said capsule endoscope to a plurality of points of a subject in a present imaging field of said imaging device, and said judging device executes a cropping process for cutting a zone of a limited subject distance range out of each endoscopic image on the basis of the distances measured by said multi-point ranging device, and analyzes image data of said zone of said endoscopic image to judge whether any area of concern exits in said zone.

9. A capsule endoscopy system as recited in claim 2, wherein said control command generating device generates a first control command for driving said capsule endoscope in a regular imaging mode when said judging device judges that no area of concern exits, whereas said control command generating device generates a second control command for driving said capsule endoscope in a special imaging mode when said judging device judges that an area of concern exits, so said capsule endoscope may capture detailed images of the area of concern in said special mode.

10. A capsule endoscopy system as recited in claim 9, wherein said capsule endoscope is driven to capture images at a higher frame rate while varying imaging conditions more widely in said special imaging mode than in said regular imaging mode.

11. A capsule endoscopy system as recited in claim 9, wherein said judging device detects a position of an area of concern within an imaging field of said imaging device after judging that the area of concern exists, and said control command generating device generates said second control command to include information on the detected position of the area of concern, whereas said capsule endoscope comprises a mechanism for directing an optical axis of said imaging device toward the area of concern in said special imaging mode according to said second control command.

12. A capsule endoscopy system as recited in claim 2, wherein said capsule endoscope comprises at least two imaging devices facing different directions from each other and a direction sensor for detecting attitude and traveling direction of said capsule endoscope, and wherein said control command generator determines respective facing directions of said imaging devices on the basis of the detected attitude and traveling direction of said capsule endoscope, and generates a first control command for driving a forward one of said imaging devices, which presently faces forward in the traveling direction, in a regular imaging mode, and when said judging device judges that an area of concern exits in an endoscopic image as captured by said forward imaging device, said control command generator generates a second control command for driving at least one of other imaging devices than said forward imaging device in a special imaging mode for capturing detailed images of the area of concern.

13. A capsule endoscopy system as recited in claim 12, wherein said capsule endoscope is driven to capture images at a higher frame rate while varying imaging conditions more widely in said special imaging mode than in said regular imaging mode.

14. A capsule endoscopy system as recited in claim 2, wherein said judging device and said control command generator are mounted in said portable apparatus, and said portable apparatus comprises a sender for sending said control commands wirelessly to said capsule endoscope.

15. A capsule endoscopy system as recited in claim 2, wherein said judging device and said control command generator are mounted in said information managing apparatus, and said information managing apparatus comprises a sender for sending said control commands wirelessly from said control command generator to said portable apparatus, whereas said portable apparatus comprises a sender for sending the endoscopic images wirelessly to said information managing apparatus after the endoscopic images are received from said capsule endoscope, and for sending said control commands wirelessly to said capsule endoscope after said control commands are received from said information managing apparatus.

16. A method of controlling operations of a capsule endoscope that is swallowed by a test body, to capture endoscopic images of internal portions of the test body and output the endoscopic images wirelessly, wherein said method comprising steps of:
analyzing each endoscopic image immediately after it is obtained by said capsule endoscope;
judging by a result obtained by said analyzing step whether said endoscopic image contains any area of concern that has different image characteristics from surrounding areas;
generating control commands for controlling operations of respective members of said capsule endoscope on the basis of a result of said judging step; and
controlling operations of the respective members of said capsule endoscope in accordance with said control commands.
